(19) United States
(12) Patent Application Publication
(10) Pub. No.: US 2024/0287928 A1
(43) Pub. Date: Aug. 29, 2024

(54) EXHAUST GAS AFTERTREATMENT SYSTEM

(71) Applicant: Cummins Emission Solutions Inc., Columbus, IN (US)

(72) Inventors: Matthew K. Volmerding, Columbus, IN (US); Varun Sood, Columbus, IN (US); Ramya vyas Arvind, Oakville (CA); James Zokoe, JR., Columbus, IN (US); Ying Yuan, Rexford, NY (US); Yuying Song, Columbus, IN (US); Rayomand Dabhoiwala, Columbus, IN (US); Shirish S. Punde, Columbus, IN (US)

(73) Assignee: Cummins Emission Solutions Inc., Columbus, IN (US)

(21) Appl. No.: 18/653,126

(22) Filed: May 2, 2024

Related U.S. Application Data
(63) Continuation of application No. PCT/US2022/048627, filed on Nov. 1, 2022.
(60) Provisional application No. 63/275,667, filed on Nov. 4, 2021.

(51) Int. Cl. F01N 13/00; B01D 53/94; B01J 29/072; B01J 35/00; B01J 35/56; F01N 3/035; F01N 3/10; F01N 3/20

(52) U.S. Cl. CPC F01N 13/009; B01D 53/9436; B01D 53/944; B01D 53/9472; B01D 53/9495; B01J 29/072; B01J 35/19; B01J 35/56; F01N 3/035; F01N 3/106; F01N 3/2066; B01D 2255/20738; B01D 2255/20761; B01D 2255/50; B01D 2255/9155; B01D 2257/302; B01D 2257/404; B01D 2257/406; B01D 2257/502; B01D 2257/702; B01D 2258/01; F01N 2510/065; F01N 2610/03; F01N 2610/1453

(57) ABSTRACT

An aftertreatment system includes an upstream particulate filter, a decomposition chamber, a decomposition chamber dosing module, a first downstream catalyst member, and a downstream particulate filter. The decomposition chamber is positioned downstream of the upstream particulate filter. The decomposition chamber dosing module is coupled to the decomposition chamber and is configured to provide downstream treatment fluid into the decomposition chamber. The first downstream catalyst member is positioned downstream of the decomposition chamber and comprises a first downstream catalyst substrate configured to facilitate treatment of exhaust gas. The downstream particulate filter positioned downstream of the first downstream catalyst member.

### CROSS-REFERENCE TO RELATED APPLICATIONS

This application is a bypass continuation of PCT Application No. PCT/US2022/048627, filed Nov. 1, 2022, which claims the benefit of and priority to U.S. Provisional Application No. 63/275,667, filed on Nov. 4, 2021. The entire disclosures of these applications are incorporated by reference herein.

### TECHNICAL FIELD

The present disclosure relates generally to an exhaust gas aftertreatment system for an internal combustion engine.

### BACKGROUND

For an internal combustion engine system, it may be desirable to reduce an amount of particulate expelled into the environment. One way to reduce the amount of particulates is to filter particulates from exhaust gas. The exhaust gas can be filtered using an aftertreatment system. One approach that can be implemented to reduce emissions is to treat the exhaust gas using an aftertreatment system. It is often desirable to mix exhaust gas with a reductant used to treat the exhaust gas. However, it can be difficult to desirably mix the reductant with the exhaust gas. For example, space constraints on the aftertreatment system can make it difficult to desirably mix the exhaust gas and the reductant in some applications.

### SUMMARY

In one embodiment, an aftertreatment system includes an upstream particulate filter, a decomposition chamber, a decomposition chamber dosing module, a first downstream catalyst member, and a downstream particulate filter. The decomposition chamber is positioned downstream of the upstream particulate filter. The decomposition chamber dosing module is coupled to the decomposition chamber and is configured to provide downstream treatment fluid into the decomposition chamber. The first downstream catalyst member is positioned downstream of the decomposition chamber and comprises a first downstream catalyst substrate configured to facilitate treatment of an exhaust gas. The downstream particulate filter positioned downstream of the first downstream catalyst member.

An aftertreatment system includes an intake chamber, an intake chamber dosing module, a decomposition chamber, a decomposition chamber dosing module, a downstream particulate filter, a first sensor, and an aftertreatment system controller. The intake chamber dosing module is coupled to the intake chamber and configured to provide an upstream treatment fluid to exhaust gas within the intake chamber. The decomposition chamber is positioned downstream of the intake chamber. The decomposition chamber dosing module is coupled to the decomposition chamber and configured to provide a downstream treatment fluid to exhaust gas within the decomposition chamber. The downstream particulate filter is positioned downstream of the decomposition chamber dosing module. The first sensor is positioned downstream of the downstream particulate filter and configured to provide a first signal. The aftertreatment system controller is communicable with the first sensor, the intake chamber dosing module, and the decomposition chamber dosing module. The aftertreatment system controller configured to cause the intake chamber dosing module to provide a first amount of the upstream treatment fluid into the intake chamber, cause the decomposition chamber dosing module to provide a second amount of the downstream treatment fluid into the decomposition chamber, receive the first signal from the first sensor, determine a first measurement based on the first signal, determine that the first measurement is greater than a first threshold, cause the intake chamber dosing module to provide a third amount of the upstream treatment fluid into the intake chamber after determining that the first measurement is greater than the first threshold, the third amount being greater than the first amount, and cause the decomposition chamber dosing module to provide a fourth amount of the downstream treatment fluid into the decomposition chamber after determining that the first measurement is greater than the first threshold, the fourth amount being less than the second amount and less than the third amount.

### BRIEF DESCRIPTION OF THE DRAWINGS

The disclosure will become more fully understood from the following detailed description, taken in conjunction with the accompanying Figures, wherein like reference numerals refer to like elements unless otherwise indicated, in which:

FIG. 1 is a cross-sectional view of a portion of an example exhaust gas aftertreatment system;

FIG. 2 is a cross-sectional view of a portion of another example exhaust gas aftertreatment system;

FIG. 3 is another cross-sectional view of a portion of another example of the exhaust gas aftertreatment system including a catalyzed filter;

FIG. 4 is a cross-sectional view of a portion of another example the exhaust gas aftertreatment system of FIG. 2;

FIG. 5 is a cross-sectional view of a portion of another example of the exhaust gas aftertreatment system of FIG. 2;

FIG. 6 is a cross-sectional view of a portion of another example of the exhaust gas aftertreatment system including a catalyzed filter.

FIG. 7 is a flow chart of an example aftertreatment system control strategy;

It will be recognized that the Figures are schematic representations for purposes of illustration. The Figures are provided for the purpose of illustrating one or more implementations with the explicit understanding that the Figures will not be used to limit the scope or the meaning of the claims.

### DETAILED DESCRIPTION

Following below are more detailed descriptions of various concepts related to, and implementations of, methods, apparatuses, and for treating exhaust gas of an internal combustion engine with an exhaust gas aftertreatment system (or simply “aftertreatment system”). The various concepts introduced above and discussed in greater detail below may be implemented in any of a number of ways, as the described concepts are not limited to any particular manner of implementation. Examples of specific implementations and applications are provided primarily for illustrative purposes.

### I. Overview

In order to reduce particulates emitted from an internal combustion engine, it may be desirable to treat exhaust gas by injecting reductant into the exhaust gas. Reduction of the emissions may be enhanced by filtering the exhaust gas within an aftertreatment system. Further reduction of the particulates within the exhaust gas may be achieved by injecting treatment fluid into the exhaust gas and having the exhaust gas flow through various catalyst members.

Various devices may be used to reduce particulates within the exhaust gas. For example, an aftertreatment system may include a plurality of filters caused to filter the exhaust gas of particulates at various locations within the aftertreatment system. However, it may be possible to further reduce particulates within the aftertreatment system by providing additional mechanisms for causing regeneration or particulate formation in the exhaust gas.

Implementations herein are directed to an aftertreatment system that includes at least one dosing module, at least one catalyst member, an upstream particulate filter assembly and a downstream particulate filter assembly. The dosing module is configured to provide treatment fluid and/or hydrocarbons in to the exhaust gas. The catalyst member is configured to react with the treatment fluid/hydrocarbons and the exhaust gas to reduce emissions. Further, the upstream particulate filter assembly includes an upstream particulate filter configured to filter particulates within the exhaust gas as the exhaust gas passes through and the downstream particulate filter assembly includes a downstream particulate filter configured to filter particulates within the exhaust gas. The upstream particulate filter and downstream particulate filter assist in reducing the number of particulates in the exhaust gas prior to being expelled.

The implementations herein directed to an aftertreatment system may provide one or more benefits including, for example: (1) reducing the particulate number within the exhaust gas below a threshold value; (2) optimization of the injection of treatment fluid by a dosing module; (3) regeneration of each of the plurality of filters within the aftertreatment system; and (4) coating each of the plurality of filters with catalyst members to reduce quantity of internal components within the aftertreatment system.

### II. Overview of Aftertreatment Systems

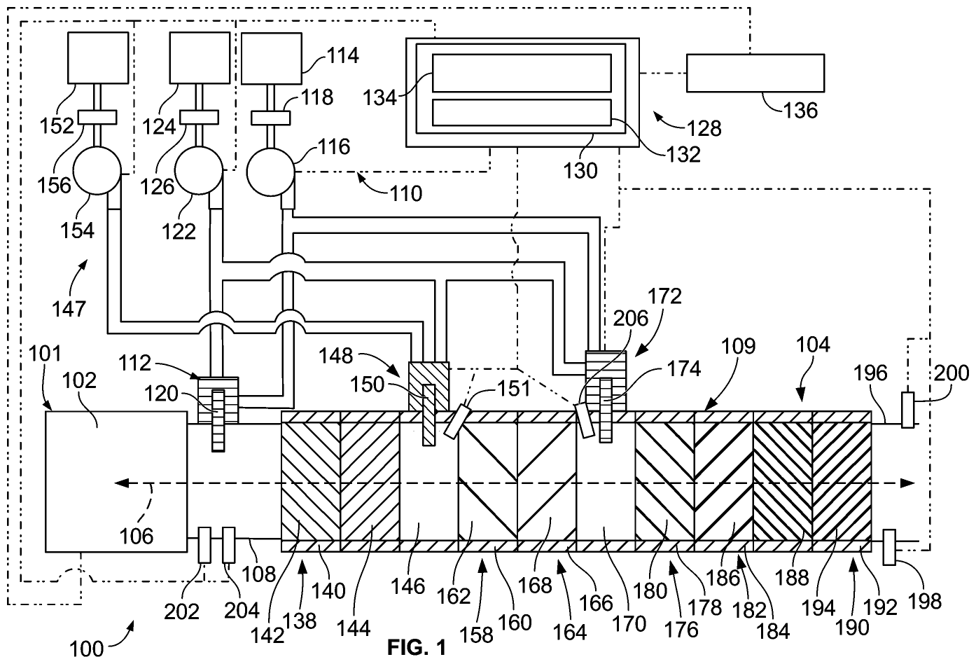

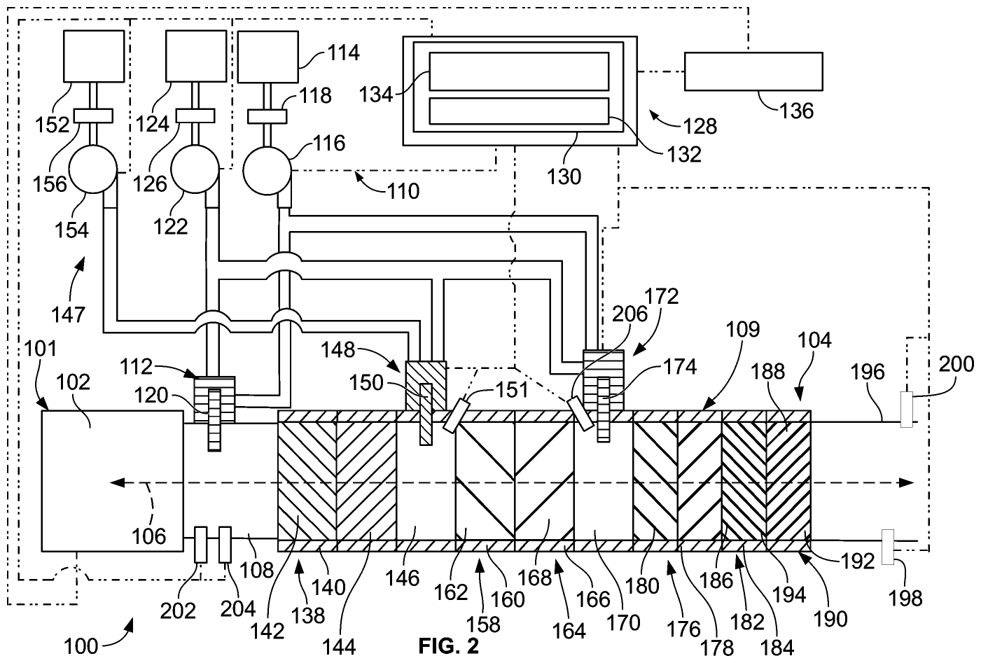

FIG. 1 and FIG. 2 depict an aftertreatment system 100 (e.g., treatment system, etc.) for an internal combustion engine system 101. The internal combustion engine system 101 includes an internal combustion engine (e.g., diesel internal combustion engine, gasoline internal combustion engine, hybrid internal combustion engine, propane internal combustion engine, dual-fuel internal combustion engine, etc.). The internal combustion engine system 101 includes an engine control component 102. The aftertreatment system 100 is configured to treat exhaust gas produced by the internal combustion engine. As is explained in more detail herein, the aftertreatment system 100 is configured to facilitate treatment of the exhaust gas. The treatment may facilitate reduction of emission of undesirable components (e.g., nitrogen oxides (NOₓ), Sulfur Oxide (SOₓ), etc.) in the exhaust gas. The treatment may also or instead facilitate conversion of various oxidation components (e.g., carbon monoxide (CO), hydrocarbons, etc.) of the exhaust gas into other components (e.g., CO₂, water vapor, etc.). The treatment may also or instead facilitate removal of particulates (e.g., soot, particulate matter, etc.) from the exhaust gas.

The aftertreatment system 100 includes an exhaust gas conduit system 104 (e.g., line system, pipe system, etc.). The exhaust gas conduit system 104 is configured to facilitate routing of the exhaust gas produced by the internal combustion engine throughout the aftertreatment system 100 and to atmosphere (e.g., ambient environment, etc.). The exhaust gas conduit system 104 is centered on a conduit center axis 106 (e.g., the conduit center axis 106 extends through a center point of the exhaust gas conduit system 304, etc.). As used herein, the term “axis” describes a theoretical line extending through the centroid (e.g., center of mass, etc.) of an object. The object is centered on the axis. The object is not necessarily cylindrical (e.g., a non-cylindrical shape may be centered on an axis, etc.).

The exhaust gas conduit system 104 includes an intake chamber 108 (e.g., line, pipe, etc.). The intake chamber 108 is configured to receive exhaust gas from the internal combustion engine. The intake chamber 108 may receive exhaust gas from a portion of the internal combustion engine (e.g., header on the internal combustion engine, exhaust manifold on the internal combustion engine, the internal combustion engine, etc.). In some embodiments, the intake chamber 108 is coupled (e.g., attached, fixed, welded, fastened, riveted, adhesively attached, bonded, pinned, press-fit, etc.) to the internal combustion engine. In other embodiments, the intake chamber 108 is integrally formed with the internal combustion engine. As utilized herein, two or more elements are “integrally formed” with each when the two or more elements are formed and joined together as part of a single manufacturing process to create a single-piece or unitary construction that cannot be disassembled without an at least partial destruction of the overall component. The intake chamber 108 may be centered on the conduit center axis 106 (e.g., the conduit center axis 106 extends through a center point of the intake chamber 108, etc.). In some embodiments, the intake chamber 108 may be offset from the conduit center axis 106 (e.g., the conduit center axis 106 extends adjacent to a center point of the intake chamber 108, etc.).

In some embodiments, the exhaust gas conduit system 104 also includes an introduction conduit 109 (e.g., decomposition housing, decomposition reactor, decomposition chamber, reactor pipe, decomposition tube, reactor tube, etc.). The introduction conduit 109 is configured to receive exhaust gas from the intake chamber 108. In various embodiments, the introduction conduit 109 is coupled to the intake chamber 108. For example, the introduction conduit 109 may be fastened (e.g., using a band, using bolts, using twist-lock fasteners, threaded, etc.), welded, riveted, or otherwise attached to the intake chamber 108. In other embodiments, the introduction conduit 109 is integrally formed with the intake chamber 108. As utilized herein, the terms “fastened,” “fastening,” and the like, describe attachment (e.g., joining, etc.) of two structures in such a way that detachment (e.g., separation, etc.) of the two structures remains possible while “fastened” or after the “fastening” is completed, without destroying or damaging either or both of the two structures. The introduction conduit 109 is centered on the conduit center axis 106 (e.g., the conduit center axis 106 extends through a center point of the introduction conduit 109, etc.). In some embodiments, the introduction conduit 109 is formed by the coupling of the individual housings and chambers, as described herein.

The aftertreatment system 100 also includes a treatment fluid delivery system 110. As is explained in more detail herein, the treatment fluid delivery system 110 is configured to facilitate the introduction of a treatment fluid, such as a reductant (e.g., diesel exhaust fluid (DEF), Adblue®, a urea-water solution (UWS), an aqueous urea solution, AUS32, etc.) or a hydrocarbon (e.g., fuel, oil, additive, etc.), into the exhaust gas within the exhaust gas. When the reductant is introduced into the exhaust gas, reduction of emission of undesirable components in the exhaust gas using the aftertreatment system 100 may be facilitated. When the hydrocarbon is introduced into the exhaust gas, the temperature of the exhaust gas may be increased (e.g., to facilitate regeneration of components of the aftertreatment system 100, etc.). For example, the temperature of the exhaust gas may be increased by combusting the hydrocarbon within the exhaust gas (e.g., using a spark plug, etc.).

The treatment fluid delivery system 110 includes an intake chamber dosing module 112 (e.g., doser, reductant doser, etc.). The intake chamber dosing module 112 is configured to facilitate passage of the treatment fluid through the intake chamber 108 and into intake chamber 108. In some embodiments, the intake chamber dosing module 112 is positioned within a dosing module mount. The dosing module mount is configured to facilitate mounting of the intake chamber dosing module 112 to the intake chamber 108. The dosing module mount may provide insulation (e.g., thermal insulation, vibrational insulation, etc.) between the intake chamber dosing module 112 and the intake chamber 108. In some embodiments, the treatment fluid delivery system 110 does not include the intake chamber dosing module 112.

The treatment fluid delivery system 110 also includes a treatment fluid source 114 (e.g., reductant tank, etc.). The treatment fluid source 114 is configured to contain the treatment fluid. The treatment fluid source 114 is configured to provide the treatment fluid to the intake chamber dosing module 112. The treatment fluid source 114 may include multiple treatment fluid sources 114 (e.g., multiple tanks connected in series or in parallel, etc.). The treatment fluid source 114 may be, for example, a diesel exhaust fluid tank containing Adblue®.

The treatment fluid delivery system 110 also includes a treatment fluid pump 116 (e.g., supply unit, etc.). The treatment fluid pump 116 is configured to receive the treatment fluid from the treatment fluid source 114 and to provide the treatment fluid to the intake chamber dosing module 112. The treatment fluid pump 116 is used to pressurize the treatment fluid from the treatment fluid source 114 for delivery to the intake chamber dosing module 112. In some embodiments, the treatment fluid pump 116 is pressure controlled. In some embodiments, the treatment fluid pump 116 is coupled to a chassis of a vehicle associated with the aftertreatment system 100.

In some embodiments, the treatment fluid delivery system 110 also includes a treatment fluid filter 118. The treatment fluid filter 118 is configured to receive the treatment fluid from the treatment fluid source 114 and to provide the treatment fluid to the treatment fluid pump 116. The treatment fluid filter 118 filters the treatment fluid prior to the treatment fluid being provided to internal components of the treatment fluid pump 116. For example, the treatment fluid filter 118 may inhibit or prevent the transmission of solids to the internal components of the treatment fluid pump 116. In this way, the treatment fluid filter 118 may facilitate prolonged desirable operation of the treatment fluid pump 116.

The intake chamber dosing module 112 includes at least one intake chamber dosing module injector 120 (e.g., insertion device, etc.). The intake chamber dosing module injector 120 configured to receive the treatment fluid from the treatment fluid pump 116. The intake chamber dosing module injector 120 is configured to dose (e.g., provide, inject, insert, etc.) the treatment fluid received by the intake chamber dosing module 112 into the exhaust gas within the intake chamber 108.

In some embodiments, the treatment fluid delivery system 110 also includes an air pump 122 and an air source 124 (e.g., air intake, etc.). The air pump 122 is configured to receive air from the air source 124. The air pump 122 is configured to provide the air to the intake chamber dosing module 112. In some applications, the intake chamber dosing module 112 is configured to mix the air and the treatment fluid into an air-treatment fluid mixture and to provide the air-treatment fluid mixture to the intake chamber dosing module injector 120 (e.g., for dosing into the exhaust gas within the intake chamber 108, etc.). As used herein, it is understood that a treatment fluid may include or consistent of an air-treatment fluid mixture.

The intake chamber dosing module injector 120 is configured to receive the air from the air pump 122. The intake chamber dosing module injector 120 is configured to dose the air into the exhaust gas within the intake chamber 108. In some of these embodiments, the treatment fluid delivery system 110 also includes an air filter 126. The air filter 126 is configured to receive the air from the air source 124 and to provide the air to the air pump 122. The air filter 126 is configured to filter the air prior to the air being provided to the air pump 122. In other embodiments, the treatment fluid delivery system 110 does not include the air pump 122 and/or the treatment fluid delivery system 110 does not include the air source 124. In such embodiments, the intake chamber dosing module 112 is not configured to mix the treatment fluid with the air.

In various embodiments, the intake chamber dosing module 112 is configured to receive air and fluid, and doses the treatment fluid into the intake chamber 108. In various embodiments, the intake chamber dosing module 112 is configured to receive treatment fluid (and does not receive air), and doses the treatment fluid into the intake chamber 108. In various embodiments, the intake chamber dosing module 112 is configured to receive treatment fluid, and doses the treatment fluid into the intake chamber 108. In various embodiments, the intake chamber dosing module 112 is configured to receive air and treatment fluid, and doses the treatment fluid into the intake chamber 108.

The aftertreatment system 100 also includes an aftertreatment system controller 128 (e.g., control circuit, driver, etc.). The intake chamber dosing module 112, the treatment fluid pump 116, and the air pump 122 are also electrically or communicatively coupled to the aftertreatment system controller 128. The aftertreatment system controller 128 is configured to control the intake chamber dosing module 112 to dose the treatment fluid into the intake chamber 108. The aftertreatment system controller 128 may also be configured to control the treatment fluid pump 116 and/or the air pump 122 in order to control the treatment fluid that is dosed into the intake chamber 108.

The aftertreatment system controller 128 includes an aftertreatment system processing circuit 130. The aftertreatment system processing circuit 130 includes an aftertreatment system processor 132 and an aftertreatment system memory 134. The aftertreatment system processor 132 may include a microprocessor, an application-specific integrated circuit (ASIC), a field-programmable gate array (FPGA), etc., or combinations thereof. The aftertreatment system memory 134 may include, but is not limited to, electronic, optical, magnetic, or any other storage or transmission device capable of providing a processor, ASIC, FPGA, etc. with program instructions. The aftertreatment system memory 134 may include a memory chip, Electrically Erasable Programmable Read-Only Memory (EEPROM), Erasable Programmable Read Only Memory (EPROM), flash memory, or any other suitable memory from which the aftertreatment system controller 128 can read instructions. The instructions may include code from any suitable programming language. The aftertreatment system memory 134 may include various modules that include instructions that are configured to be implemented by the aftertreatment system processor 132.

In various embodiments, the aftertreatment system controller 128 is configured to communicate with a central controller 136 (e.g., engine control unit (ECU), engine control module (ECM), etc.) to control the engine control component 102. The engine control component 102 is configured to control the operation of a component of the internal combustion engine (e.g., throttle valve, fuel valve, air valve, ignition timing circuit, etc.). In some embodiments, the central controller 136 and the aftertreatment system controller 128 are integrated into a single controller.

In some embodiments, the central controller 136 is communicable with a display device (e.g., screen, monitor, touch screen, heads up display (HUD), indicator light, etc.). The display device may be configured to change state in response to receiving information from the central controller 136. For example, the display device may be configured to change between a static state and an alarm state based on a communication from the central controller 136. By changing state, the display device may provide an indication to a user of a status of the treatment fluid delivery system 110.

The aftertreatment system 100 includes an upstream catalyst member 138 (e.g., conversion catalyst member, selective catalytic reduction (SCR) catalyst member, catalyst metals, etc.). The upstream catalyst member 138 is positioned downstream of the intake chamber 108. The upstream catalyst member 138 is configured to cause decomposition of components of the exhaust gas using the treatment fluid (e.g., via catalytic reactions, etc.). The upstream catalyst member 138 includes an upstream catalyst housing 140. The upstream catalyst housing 140 may be coupled to the intake chamber 108. In some embodiments, the upstream catalyst housing 140 is integrally formed with the intake chamber 108. The upstream catalyst member 138 includes an upstream catalyst substrate 142. The upstream catalyst substrate 142 is coupled to the upstream catalyst housing 140. In some embodiments, the upstream catalyst substrate 142 is integrally formed with the upstream catalyst housing 140.

The upstream catalyst member 138 receives the exhaust gas from the intake chamber 108. The exhaust gas flows through the upstream catalyst substrate 142 and reacts with the upstream catalyst substrate 142 so as to cause the exhaust gas to undergo the processes of evaporation, thermolysis, and/or hydrolysis to form non-NOₓ emissions within the introduction conduit 109 and/or the upstream catalyst member 138. In some embodiments, the exhaust gas and the treatment fluid within the exhaust gas react with the upstream catalyst substrate 142. In this way the upstream catalyst member 138 is configured to assist the reduction of NOₓ emissions by accelerating a NOₓ reduction process between the reductant and the NOₓ of the exhaust gas into diatomic nitrogen, water, and/or carbon dioxide. The upstream catalyst substrate 142 may include vanadia. Vanadia is used due to the lengthy deactivation time and the ability to react with the exhaust gas at high temperatures. In some embodiments, vanadia is used because of the benefit of emitting lower N₂O emissions into the environment when exhaust gas temperatures are below 420° C. In some embodiments, the aftertreatment system 100 does not include an upstream catalyst member 138.

The aftertreatment system 100 includes an upstream ammonia slip catalyst substrate 144. The upstream ammonia slip catalyst substrate 144. The upstream ammonia slip catalyst substrate 144 is positioned downstream of the upstream catalyst member 138. In some embodiments, the upstream ammonia slip catalyst substrate 144 is a coating applied to a portion of the outlet of the upstream catalyst member 138. The upstream ammonia slip catalyst substrate 144 is configured to receive the exhaust gas from the upstream catalyst member 138 and assist in the reduction of the byproducts (e.g., ammonia, etc.) of the processes of the intake chamber dosing module 112 and the upstream catalyst member 138. Specifically, the intake chamber dosing module 112 may introduce ammonia into the exhaust gas, however a portion of the ammonia introduced may not react with the exhaust gas. As a result, excess ammonia may slip from the upstream catalyst member 138 into the exhaust gas downstream of the upstream catalyst member 138. The upstream ammonia slip catalyst substrate 144 functions to reduce the ammonia such that the exhaust gas downstream of the upstream ammonia slip catalyst substrate 144 does not contain an undesirable amount of ammonia. In some embodiments, the aftertreatment system 100 does not include the upstream ammonia slip catalyst substrate 144.

The aftertreatment system 100 also includes a first hydrocarbon decomposition chamber 146. The first hydrocarbon decomposition chamber is positioned downstream of the upstream ammonia slip catalyst substrate 144. In some embodiments the first hydrocarbon decomposition chamber 146 is coupled to the upstream catalyst housing 140. In some embodiments, the first hydrocarbon decomposition chamber 146 is integrally formed with the upstream catalyst housing 140. In still other embodiments, the first hydrocarbon decomposition chamber 146 is coupled to the intake chamber 108. In some embodiments, the aftertreatment system 100 does not include the upstream catalyst member 138 such that the first hydrocarbon decomposition chamber 146 may also be integrally formed with the intake chamber 108. The first hydrocarbon decomposition chamber 146 is configured to receive the exhaust gas from the upstream ammonia slip catalyst substrate 144. The aftertreatment system 100 includes a hydrocarbon fluid system 147. The hydrocarbon fluid system 147 includes a first hydrocarbon dosing module 148. The first hydrocarbon dosing module 148 doses the exhaust gas within the first hydrocarbon decomposition chamber 146 with hydrocarbons. The first hydrocarbon dosing module 148 is configured to facilitate passage of hydrocarbon through the first hydrocarbon decomposition chamber 146 and into the first hydrocarbon decomposition chamber 146. The first hydrocarbon dosing module 148 includes at least one first hydrocarbon injector 150 (e.g., insertion device, etc.). The first hydrocarbon injector 150 is configured to dose the hydrocarbons into the exhaust gas within the first hydrocarbon decomposition chamber 146.

The hydrocarbons within the first hydrocarbon decomposition chamber 146 may be configured to increase the temperature of the exhaust gas within the first hydrocarbon decomposition chamber 146. Specifically, the aftertreatment system 100 includes an igniter 151 (e.g., spark plug, etc.) coupled to the first hydrocarbon decomposition chamber 146. The igniter 151 is electrically connected to the aftertreatment system controller 128 and is configured to combust the hydrocarbons in the exhaust gas within the first hydrocarbon decomposition chamber 146 which causes the increase in temperature of the exhaust gas. By this way, regeneration of downstream components may occur.

The hydrocarbon fluid system 147 further includes a hydrocarbon source 152 (e.g., hydrocarbon tank, etc.). The hydrocarbon source 152 is configured to contain hydrocarbon. The hydrocarbon source 152 is configured to provide hydrocarbons to the first hydrocarbon dosing module 148. The hydrocarbon source 152 may include multiple hydrocarbon sources 152 (e.g., multiple tanks connected in series or in parallel, etc.). The treatment fluid delivery system also includes a hydrocarbon pump 154. Specifically, the hydrocarbon pump 154 is configured to provide hydrocarbon to the first hydrocarbon injector 150. The first hydrocarbon injector 150 receives hydrocarbon from the hydrocarbon pump 154 and is configured to dose the hydrocarbon received by the first hydrocarbon dosing module 148 into the exhaust gas within the first hydrocarbon decomposition chamber 146. The hydrocarbon pump 154 is used to pressurize hydrocarbons from the hydrocarbon source 152 for delivery to the first hydrocarbon dosing module 148 and the first hydrocarbon injector 150. In some embodiments, the hydrocarbon pump 154 is pressure controlled. In some embodiments the hydrocarbon pump 154 is coupled to a chassis of a vehicle associated with the aftertreatment system.

In some embodiments, the hydrocarbon fluid system 147 includes a hydrocarbon filter 156 (e.g., fuel filter, lubricant filter, oil filter, etc.). The hydrocarbon filter 156 is configured to receive the hydrocarbons from the hydrocarbon source 152 and to provide the hydrocarbons to the hydrocarbon pump 154. The hydrocarbon filter 156 filters the hydrocarbons prior to the hydrocarbons being provided to internal components of the hydrocarbon pump 154. For example, the hydrocarbon filter 156 may inhibit or prevent the transmission of solids to the internal components of the hydrocarbon pump 154. In this way, the hydrocarbon filter 156 may facilitate prolonged desirable operation of the hydrocarbon pump 154.

In some embodiments, the air pump 122 is also configured to provide the air to the first hydrocarbon dosing module 148. The first hydrocarbon dosing module 148 is configured to provide the air into the first hydrocarbon decomposition chamber 146. In some applications, the first hydrocarbon dosing module 148 is configured to mix the air and the hydrocarbons into an air-hydrocarbon fluid mixture and to provide the air-hydrocarbon fluid mixture to the first hydrocarbon injector 150 (e.g., for dosing into the exhaust gas within the first hydrocarbon decomposition chamber 146, etc.).

In various embodiments, the first hydrocarbon dosing module 148 is configured to receive air and hydrocarbon, and doses the hydrocarbons into the first hydrocarbon decomposition chamber 146. In various embodiments, the first hydrocarbon dosing module 148 is configured to receive hydrocarbons (and does not receive air), and doses the hydrocarbon into the intake chamber 108. In various embodiments, the first hydrocarbon dosing module 148 is configured to receive hydrocarbons, and doses the hydrocarbon into the first hydrocarbon decomposition chamber 146.

In some embodiments, the first hydrocarbon dosing module 148 and the hydrocarbon pump 154 are also electrically or communicatively coupled to the aftertreatment system controller 128. The aftertreatment system controller 128 is further configured to control the first hydrocarbon dosing module 148 to dose the hydrocarbon into the first hydrocarbon decomposition chamber 146. The aftertreatment system controller 128 may also be configured to control the hydrocarbon pump 154 and/or the air pump 122 in order to control the hydrocarbon that is dosed into the first hydrocarbon decomposition chamber 146. In some embodiments, the aftertreatment system 100 does not include the first hydrocarbon decomposition chamber 146, the first hydrocarbon dosing module 148, the first hydrocarbon injector 150, the hydrocarbon source 152, the hydrocarbon pump 154, and/or the hydrocarbon filter 156.

The aftertreatment system 100 includes a first oxidation catalyst member 158 (e.g., first diesel oxidation catalyst (DOC), etc.). The first oxidation catalyst member 158 is positioned downstream of first hydrocarbon decomposition chamber 146 (i.e., the first hydrocarbon decomposition chamber 146 is positioned upstream of the first oxidation catalyst member 158).

The first oxidation catalyst member 158 includes a first oxidation catalyst housing 160. The first oxidation catalyst housing 160 is coupled to first hydrocarbon decomposition chamber 146. The first oxidation catalyst housing 160 may also be integrally formed with the first hydrocarbon decomposition chamber 146.

The first oxidation catalyst member 158 also includes a first oxidation catalyst substrate 162 (e.g., DOC, etc.). The first oxidation catalyst substrate 162 is positioned within the first oxidation catalyst housing 160. The first oxidation catalyst substrate 162 may be coupled to the first oxidation catalyst housing 160. The exhaust gas including hydrocarbons react with the first oxidation catalyst substrate 162 and cause the conversion hydrocarbons in the exhaust gas. For example, as the exhaust gas flows the through the first oxidation catalyst substrate 162, the hydrocarbons react with the first oxidation catalyst substrate 162 and began to oxidize. The first oxidation catalyst substrate 162 facilitates conversion of the carbon monoxide in the exhaust gas and the hydrocarbons and/or the air-hydrocarbon mixture into carbon dioxide.

In some embodiments, the aftertreatment system 100 does not include the upstream catalyst member 138, the upstream ammonia slip catalyst substrate 144, and/or the first hydrocarbon decomposition chamber 146 such that the first oxidation catalyst member 158 may be positioned downstream of the intake chamber 108. In such embodiments, the first oxidation catalyst housing 160 may be coupled to the intake chamber 108. In other such embodiments, the first oxidation catalyst housing 160 may also be integrally formed with the intake chamber 108.

The aftertreatment system 100 also includes an upstream particulate filter assembly 164. The upstream particulate filter assembly 164 includes an upstream particulate filter housing 166. The upstream particulate filter housing 166 is positioned downstream of the first oxidation catalyst housing 160. In some embodiments, the upstream particulate filter housing 166 is integrally formed with the first oxidation catalyst housing 160. The upstream particulate filter assembly 164 includes an upstream particulate filter 168 (e.g., diesel particulate filter (DPF), filtration member, etc.). The upstream particulate filter 168 is disposed within the upstream particulate filter housing 166 such that the upstream particulate filter 168 is positioned downstream of the first oxidation catalyst member 158 (i.e., the first oxidation catalyst member 158 is positioned upstream of the upstream particulate filter 168). In some embodiments, the upstream particulate filter housing 166 and the upstream particulate filter 168 are positioned downstream of the intake chamber 108.

The upstream particulate filter 168 is configured to remove first particulates (e.g., soot, solidified hydrocarbons, ash, etc.) from the exhaust gas. For example, the upstream particulate filter 168 may receive exhaust gas (e.g., from the first oxidation catalyst member 158, from the intake chamber 108, etc.) having a first concentration of the first particulates and may provide the exhaust gas downstream having a second concentration of the first particulates, where the second concentration is lower than the first concentration. In this way, the upstream particulate filter 168 may facilitate reduction of a particulate number (PN) of the exhaust gas. Decreasing the PN of the exhaust gas may be desirable in a variety of applications. For example, emissions regulations may prescribe a maximum PN for exhaust gas emitted to atmosphere and the upstream particulate filter 168 may ensure that the PN of the exhaust gas emitted to atmosphere by the aftertreatment system 100 is below the maximum PN.

In some embodiments, the upstream particulate filter 168 is a catalyzed DPF. The catalyzed DPF is a filter that has a catalyst coating. The catalyst coating is configured to react with a component of the exhaust gas to reduce undesirable components in the exhaust. For example, the catalyst coating could be an oxidation catalyst to reduce hydrocarbons within the exhaust gas. In some embodiments, the catalyst coating is a SCR catalyst configured to reduce NOₓ emissions.

The aftertreatment system 100 also includes a decomposition chamber 170 (e.g., decomposition reactor, decomposition reactor tube (DRT), etc.). The decomposition chamber 170 is positioned downstream of the upstream particulate filter assembly 164 (i.e., the decomposition chamber 170 is positioned downstream of the upstream particulate filter 168) and configured to receive exhaust gas from the upstream particulate filter assembly 164. The decomposition chamber 170 may be coupled to the upstream particulate filter housing 166. In some embodiments, the decomposition chamber 170 is integrally formed with the upstream particulate filter housing 166.

The treatment fluid delivery system 110 includes a decomposition chamber dosing module 172. The decomposition chamber dosing module 172 is configured to facilitate passage of the treatment fluid through the decomposition chamber 170 and into the decomposition chamber 170. In some embodiments, the decomposition chamber dosing module 172 is positioned within a dosing module mount. The dosing module mount is configured to facilitate mounting of the decomposition chamber dosing module 172 to the decomposition chamber 170. The dosing module mount may provide insulation (e.g., thermal insulation, vibrational insulation, etc.) between the decomposition chamber dosing module 172 and the decomposition chamber.

The decomposition chamber dosing module 172 includes at least one decomposition chamber injector 174 (e.g., insertion device, etc.). The decomposition chamber injector 174 is configured to receive the treatment fluid from the treatment fluid pump 116. The decomposition chamber injector 174 is configured to dose the treatment fluid received by the decomposition chamber dosing module 172 into the exhaust gas within the decomposition chamber 170.

In some embodiments, the decomposition chamber dosing module 172 is configured to receive air from the air pump 122. In some applications, the decomposition chamber dosing module 172 is configured to mix the air and the treatment fluid into an air-treatment fluid mixture and to provide the air-treatment fluid mixture to the decomposition chamber injector 174 (e.g., for dosing into the exhaust gas within the decomposition chamber 170, etc.). Specifically, the decomposition chamber injector 174 is configured to receive the air from the air pump 122. The intake chamber dosing module injector 120 is configured to dose the air into the exhaust gas within the decomposition chamber 170.

In various embodiments, the decomposition chamber dosing module 172 is configured to receive air and fluid, and doses the treatment fluid into the decomposition chamber 170. In various embodiments, the decomposition chamber dosing module 172 is configured to receive treatment fluid (and does not receive air), and doses the treatment fluid into the decomposition chamber 170. In various embodiments, the decomposition chamber dosing module 172 is configured to receive treatment fluid, and doses the treatment fluid into the decomposition chamber 170. In various embodiments, the decomposition chamber dosing module 172 is configured to receive air and treatment fluid, and doses the treatment fluid into the decomposition chamber 170.

The aftertreatment system 100 includes a first downstream catalyst member 176 (e.g., conversion catalyst member, SCR catalyst member, catalyst metals, etc.). The first downstream catalyst member 176 is positioned downstream of the decomposition chamber 170. The first downstream catalyst member 176 is configured to cause decomposition of components of the exhaust gas using the treatment fluid (e.g., via catalytic reactions, etc.). The first downstream catalyst member 176 includes a first downstream catalyst housing 178 and a first downstream catalyst substrate 180. The first downstream catalyst housing 178 may be coupled to the decomposition chamber 170. In some embodiments, the first downstream catalyst housing 178 is integrally formed with the decomposition chamber 170. The first downstream catalyst substrate 180 is coupled to the first downstream catalyst housing 178. In some embodiments, the first downstream catalyst substrate 180 is integrally formed with the first downstream catalyst housing 178.

The first downstream catalyst member 176 receives the exhaust gas from the decomposition chamber 170. The exhaust gas flows through the first downstream catalyst substrate 180 and reacts with the first downstream catalyst substrate 180 so as to cause the exhaust gas to undergo the processes of evaporation, thermolysis, and/or hydrolysis to form non-NOₓ emissions within the introduction conduit 109 and/or the first downstream catalyst member 176. In some embodiments, the exhaust gas and the treatment fluid within the exhaust gas react with the first downstream catalyst substrate 180. In this way the first downstream catalyst member 176 is configured to assist the reduction of NOₓ emissions by accelerating a NOₓ reduction process between the reductant and the NOₓ of the exhaust gas into diatomic nitrogen, water, and/or carbon dioxide and also configured to assist in the reduction of particulates from the exhaust gas. The first downstream catalyst member 176 may include iron zeolite. The first downstream catalyst member 176 may include copper zeolite. In some embodiments, the aftertreatment system 100 does not include a first downstream catalyst member 176.

The aftertreatment system 100 also includes a second downstream catalyst member 182 (e.g., conversion catalyst member, SCR catalyst member, catalyst metals, etc.). The second downstream catalyst member 182 is positioned downstream of the decomposition chamber 170. The second downstream catalyst member 182 is configured to cause decomposition of components of the exhaust gas using the treatment fluid (e.g., via catalytic reactions, etc.). The second downstream catalyst member 182 includes a second downstream catalyst housing 184. In some embodiments, the second downstream catalyst housing 184 is integrally formed with the first downstream catalyst housing 178. In some embodiments, the second downstream catalyst housing 184 is the first downstream catalyst housing 178. The second downstream catalyst member 182 includes a second downstream catalyst substrate 186. The second downstream catalyst substrate 186 is coupled to the second downstream catalyst housing 184. In some embodiments, the second downstream catalyst substrate 186 is integrally formed with the second downstream catalyst housing 184.

The second downstream catalyst member 182 receives the exhaust gas from the first downstream catalyst member 176. The exhaust gas flows through the second downstream catalyst substrate 186 and reacts with the second downstream catalyst substrate 186 so as to cause the exhaust gas to undergo the processes of evaporation, thermolysis, and/or hydrolysis to form non-NOₓ emissions within the introduction conduit 109 and/or the second downstream catalyst member 182. In some embodiments, the exhaust gas and the treatment fluid within the exhaust gas react with the second downstream catalyst substrate 186. In this way the second downstream catalyst member 182 is configured to assist the reduction of NOₓ emissions by accelerating a NOₓ reduction process between the reductant and the NOₓ of the exhaust gas into diatomic nitrogen, water, and/or carbon dioxide and also configured to assist in the reduction of particulates from the exhaust gas. The second downstream catalyst member 182 may include iron zeolite. The second downstream catalyst member 182 may include copper zeolite. In some embodiments, the aftertreatment system 100 does not include a second downstream catalyst member 182.

The aftertreatment system 100 includes a downstream ammonia slip catalyst substrate 188. The downstream ammonia slip catalyst substrate 188 is positioned downstream of the second downstream catalyst member 182. In some embodiments, the downstream ammonia slip catalyst substrate 188 is a coating applied to a portion of the outlet of the first downstream catalyst member 176. The downstream ammonia slip catalyst substrate 188 may be a coating applied to a portion of the outlet of the second downstream catalyst member 182. The downstream ammonia slip catalyst substrate 188 is configured to receive the exhaust gas from the second downstream catalyst member 182 and assist in the reduction of the byproducts (e.g., ammonia, etc.) of the processes of the decomposition chamber dosing module 172 and the second downstream catalyst member 182. In some embodiments, the downstream ammonia slip catalyst substrate 188 is positioned downstream of the first downstream catalyst member 176 and is configured to receive the exhaust gas from the first downstream catalyst member 176 and assist in the reduction of the byproducts (e.g., ammonia, etc.) of the processes of the decomposition chamber dosing module 172 and the first downstream catalyst member 176. Specifically, the decomposition chamber dosing module 172 may introduce ammonia into the exhaust gas, however a portion of the ammonia introduced may not react with the exhaust gas. As a result, excess ammonia may slip from the first downstream catalyst member 176 and/or the second downstream catalyst member 182 into the exhaust gas downstream of the first downstream catalyst member 176 and/or the second downstream catalyst member 182 such that the exhaust gas downstream of the downstream ammonia slip catalyst substrate 188 does not contain an undesirable amount of ammonia. In some embodiments, the aftertreatment system 100 does not include the downstream ammonia slip catalyst substrate 188.

The aftertreatment system 100 also includes a downstream particulate filter assembly 190. The downstream particulate filter assembly 190 includes a downstream particulate filter housing 192 and a downstream particulate filter 194 (e.g., DPF, filtration member, etc.). The downstream particulate filter housing 192 is positioned downstream of the second downstream catalyst member 182. In some embodiments, the downstream particulate filter housing 192 is integrally formed with the second downstream catalyst housing 184. The downstream particulate filter 194 is disposed within the downstream particulate filter housing 192 such that the downstream particulate filter 194 is positioned downstream of the second downstream catalyst member 182.

In some embodiments, the downstream particulate filter housing 192 is positioned downstream of the first downstream catalyst member 176. In some embodiments, the downstream particulate filter housing 192 is integrally formed with the first downstream catalyst housing 178. The downstream particulate filter 194 is disposed within the downstream particulate filter housing 192 such that the downstream particulate filter 194 is positioned downstream of the first downstream catalyst member 176. The downstream particulate filter 194 is configured to remove second particulates from the exhaust gas. For example, the downstream particulate filter 194 may receive exhaust gas (e.g., from the first downstream catalyst member 176, the second downstream catalyst member 182, etc.) having a first concentration of the second particulates and may provide the exhaust gas downstream having a second concentration of the second particulates, where the second concentration is lower than the first concentration. In this way, the downstream particulate filter 194 may facilitate reduction of the particulate number within the exhaust gas Decreasing the PN of the exhaust gas may be desirable in a variety of applications. For example, emissions regulations may prescribe a maximum PN for exhaust gas emitted to atmosphere and the upstream particulate filter 168 may ensure that the PN of the exhaust gas emitted to atmosphere by the aftertreatment system 100 is below the maximum PN.

In some embodiments, the downstream particulate filter 194 is a catalyzed DPF. The catalyzed DPF is a filter that has a catalyst coating. The catalyst coating is configured to react with a component of the exhaust gas to reduce undesirable components in the exhaust. For example, the catalyst coating could be an oxidation catalyst to reduce hydrocarbons within the exhaust gas. In some embodiments, the catalyst coating is a SCR catalyst configured to reduce NOₓ emissions.

Referring to FIG. 2, in some embodiments, the downstream particulate filter 194 is a catalyzed filtration member including a catalyst substrate. For example the catalyst substrate such that the downstream ammonia slip catalyst substrate 188 is disposed on the downstream particulate filter 194. For example, a portion of the inlet of the downstream particulate filter 194 and a portion of the outlet of the downstream particulate filter 194 is coated with the downstream ammonia slip catalyst substrate 188. By this, the downstream particulate filter 194 coated with the downstream ammonia slip catalyst substrate 188 is configured to receive the exhaust gas and assist in byproducts of the (e.g., ammonia, etc.) of the processes of the decomposition chamber dosing module 172 and the first downstream catalyst member 176 and also remove second particulates within the exhaust gas. Specifically, the decomposition chamber dosing module 172 may introduce ammonia into the exhaust gas, however a portion of the ammonia introduced may not react with the exhaust gas. As a result, excess ammonia may slip from the, the decomposition chamber 170, the first downstream catalyst member 176 and/or the second downstream catalyst member 182 into the exhaust gas downstream of the second downstream catalyst member 182. The downstream particulate filter 194 coated with the downstream ammonia slip catalyst substrate 188 functions to reduce the ammonia such that the exhaust gas downstream of downstream particulate filter 194 does not contain an undesirable amount of ammonia.

The aftertreatment system 100 also includes an outlet chamber 196. The outlet chamber 196 is positioned downstream of the downstream particulate filter assembly 190 and is configured to receive the exhaust gas from the downstream particulate filter 194. In various embodiments, the outlet chamber 196 is coupled to the downstream particulate filter housing 192. For example, the outlet chamber 196 may be fastened, welded, riveted, or otherwise attached to the downstream particulate filter housing 192. In other embodiments, the outlet chamber 196 is integrally formed with the downstream particulate filter housing 192. In some embodiments, the outlet chamber 196 is coupled to the introduction conduit 109. In some embodiments, the outlet chamber 196 is the introduction conduit 109 (e.g., only the introduction conduit is included in the exhaust gas conduit system 104 and the introduction conduit 109 functions as both the introduction conduit 109 and the outlet chamber 196). The outlet chamber 196 is centered on the conduit center axis 106 (e.g., the conduit center axis 106 extends through a center point of the outlet chamber 196, etc.).

In various embodiments, the exhaust gas conduit system 104 only includes a single conduit that functions as the intake chamber 108, the introduction conduit 109, and the outlet chamber 196.

In various embodiments, the aftertreatment system 100 also includes a first sensor 198 (e.g., NOₓ sensor, CO sensor, CO₂ sensor, O₂ sensor, particulate sensor, nitrogen sensor, etc.). The first sensor 198 is positioned downstream of the downstream particulate filter 194. In some embodiments, the first sensor 198 is coupled to the outlet chamber 196. The first sensor 198 is configured to measure (e.g., sense, detect, etc.) a parameter (e.g., NOₓ concentration, CO concentration, CO₂ concentration, O₂ concentration, particulate concentration, nitrogen concentration, sulfur oxide concentration (SOₓ), etc.) of the exhaust gas and the treatment fluid downstream of the downstream particulate filter 194. The first sensor 198 may be configured to measure the parameter within the outlet chamber 196. In some embodiments, the parameter measured by the first sensor 198 is the particulate concentration in the exhaust gas downstream of the downstream particulate filter 194. In some embodiments, the parameter measured by the first sensor 198 is the SOₓ concentration of the exhaust gas within the outlet chamber 196. In some embodiments, the first sensor 198 measures both the particulate concentration and the SOₓ concentration.

The first sensor 198 is electrically or communicatively coupled to the aftertreatment system controller 128 and is configured to provide a first signal associated with the parameter to the aftertreatment system controller 128. The aftertreatment system controller 128 (e.g., via the aftertreatment system processing circuit 130, etc.) is configured to determine a first measurement based on the first signal. The aftertreatment system controller 128 may be configured to control the intake chamber dosing module 112, the decomposition chamber dosing module 172, the treatment fluid pump 116, and/or the air pump 122 based on the first signal. Furthermore, the aftertreatment system controller 128 may be configured to communicate the first signal to the central controller 136.

In various embodiments, the aftertreatment system 100 also includes a second sensor 200 (e.g., NOₓ sensor, CO sensor, CO₂ sensor, O₂ sensor, particulate sensor, nitrogen sensor, etc.). The second sensor 200 is positioned downstream of the downstream particulate filter 194. In some embodiments, the second sensor 200 is coupled to the outlet chamber 196 and positioned downstream of the first sensor 198. The second sensor 200 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid downstream of the downstream particulate filter 194. The second sensor 200 may be configure to measure the parameter of the exhaust gas within the outlet chamber 196. In some embodiments, the parameter measured by the second sensor 200 is the particulate concentration in the exhaust gas downstream of the downstream particulate filter 194. In some embodiments, the parameter measured by the second sensor 200 is the SOₓ concentration of the exhaust gas downstream of the downstream particulate filter 194. In some embodiments, the second sensor 200 measures both the particulate concentration and the SOₓ concentration.

The second sensor 200 is electrically or communicatively coupled to the aftertreatment system controller 128 and is configured to provide a second signal associated with the parameter to the aftertreatment system controller 128. The aftertreatment system controller 128 (e.g., via the aftertreatment system processing circuit 130, etc.) is configured to determine a second measurement based on the second signal. The aftertreatment system controller 128 may be configured to control the intake chamber dosing module 112, the decomposition chamber dosing module 172, the treatment fluid pump 116, and/or the air pump 122 based on the second signal. Furthermore, the aftertreatment system controller 128 may be configured to communicate the second signal to the central controller 136.

In various embodiments, the aftertreatment system 100 also includes a third sensor 202 (e.g., NOₓ sensor, CO sensor, CO₂ sensor, O₂ sensor, particulate sensor, nitrogen sensor, etc.). The third sensor 202 is positioned upstream of the upstream particulate filter 168. In some embodiments, the third sensor 202 is coupled to the intake chamber 108. The third sensor 202 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid upstream of the upstream particulate filter 168. The third sensor 202 may be configured to measure a parameter of the exhaust gas within the intake chamber 108. In some embodiments, the parameter measured by the first sensor 198 is the particulate concentration in the exhaust gas upstream of the upstream particulate filter 168. In some embodiments, the parameter measured by the third sensor 202 is the SOₓ concentration of the exhaust gas upstream of the upstream particulate filter 168. In some embodiments, the third sensor 202 measures both the particulate concentration and the SOₓ concentration.

The third sensor 202 is electrically or communicatively coupled to the aftertreatment system controller 128 and is configured to provide a third signal associated with the parameter to the aftertreatment system controller 128. The aftertreatment system controller 128 (e.g., via the aftertreatment system processing circuit 130, etc.) is configured to determine a third measurement based on the third signal. The aftertreatment system controller 128 may be configured to control the intake chamber dosing module 112, the decomposition chamber dosing module 172, the treatment fluid pump 116, and/or the air pump 122 based on the third signal. Furthermore, the aftertreatment system controller 128 may be configured to communicate the third signal to the central controller 136.

In various embodiments, the aftertreatment system 100 also includes a fourth sensor 204 (e.g., NOₓ sensor, CO sensor, CO₂ sensor, O₂ sensor, particulate sensor, nitrogen sensor, etc.). The fourth sensor 204 is positioned upstream of the upstream particulate filter 168. In some embodiments, the fourth sensor 204 is coupled to the intake chamber 108 and positioned downstream of the third sensor 202. The fourth sensor 204 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid upstream of the upstream particulate filter 168. The fourth sensor 204 may be configured to measure a parameter of the exhaust gas upstream of the upstream particulate filter 168. In some embodiments, the parameter measured by the fourth sensor 204 is the particulate concentration in the exhaust gas upstream of the upstream particulate filter 168. In some embodiments, the parameter measured by the fourth sensor 204 is the SOₓ concentration of the exhaust gas upstream of the upstream particulate filter 168. In some embodiments, the fourth sensor 204 measures both the particulate concentration and the SOₓ concentration.

The fourth sensor 204 is electrically or communicatively coupled to the aftertreatment system controller 128 and is configured to provide a fourth signal associated with the parameter to the aftertreatment system controller 128. The aftertreatment system controller 128 (e.g., via the aftertreatment system processing circuit 130, etc.) is configured to determine a fourth measurement based on the fourth signal. The aftertreatment system controller 128 may be configured to control the intake chamber dosing module 112, the decomposition chamber dosing module 172, the treatment fluid pump 116, and/or the air pump 122 based on the fourth signal. Furthermore, the aftertreatment system controller 128 may be configured to communicate the third signal to the central controller 136.

In various embodiments, the aftertreatment system 100 also includes a fifth sensor 206 (e.g., NOₓ sensor, CO sensor, CO₂ sensor, O₂ sensor, particulate sensor, nitrogen sensor, etc.). The fifth sensor 206 is positioned downstream of the upstream particulate filter 168. In some embodiments, the fifth sensor 206 is coupled to the decomposition chamber 170 and positioned downstream of the fourth sensor 204. The fifth sensor 206 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid downstream of the upstream particulate filter 168. The fifth sensor 206 may be configured to measure a parameter of the exhaust gas downstream of the upstream particulate filter 168. In some embodiments, the parameter measured by the fifth sensor 206 is the particulate concentration in the exhaust gas downstream of the upstream particulate filter 168. In some embodiments, the parameter measured by the fifth sensor 206 is the SOₓ concentration of the exhaust gas downstream of the upstream particulate filter 168. In some embodiments, the fifth sensor 206 measures both the particulate concentration and the SOₓ concentration.

The fifth sensor 206 is electrically or communicatively coupled to the aftertreatment system controller 128 and is configured to provide a fifth signal associated with the parameter to the aftertreatment system controller 128. The aftertreatment system controller 128 (e.g., via the aftertreatment system processing circuit 130, etc.) is configured to determine a fifth measurement based on the fifth signal. The aftertreatment system controller 128 may be configured to control the intake chamber dosing module 112, the decomposition chamber dosing module 172, the treatment fluid pump 116, and/or the air pump 122 based on the fifth signal. Furthermore, the aftertreatment system controller 128 may be configured to communicate the fifth signal to the central controller 136.

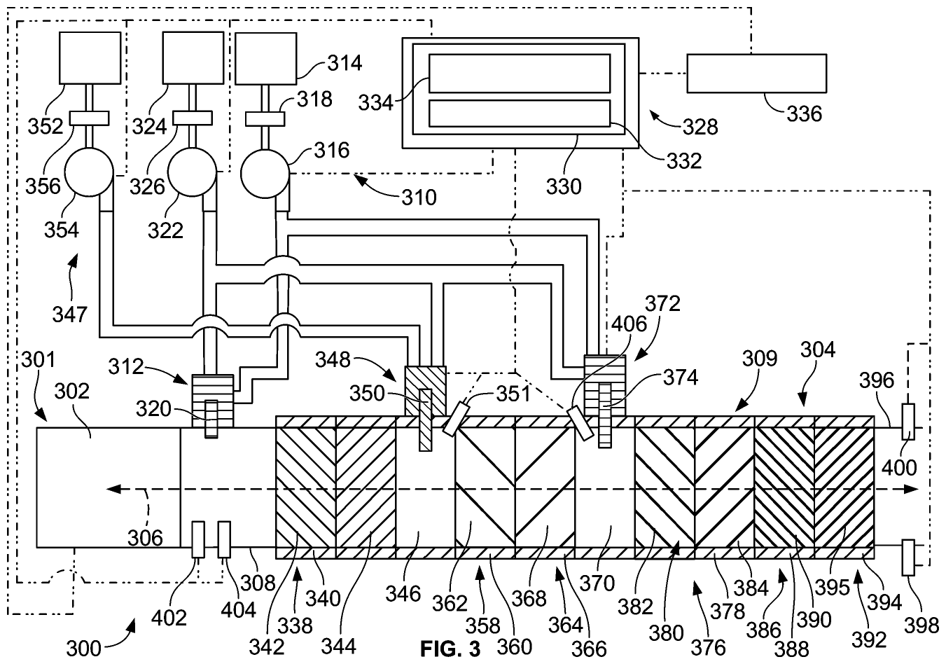

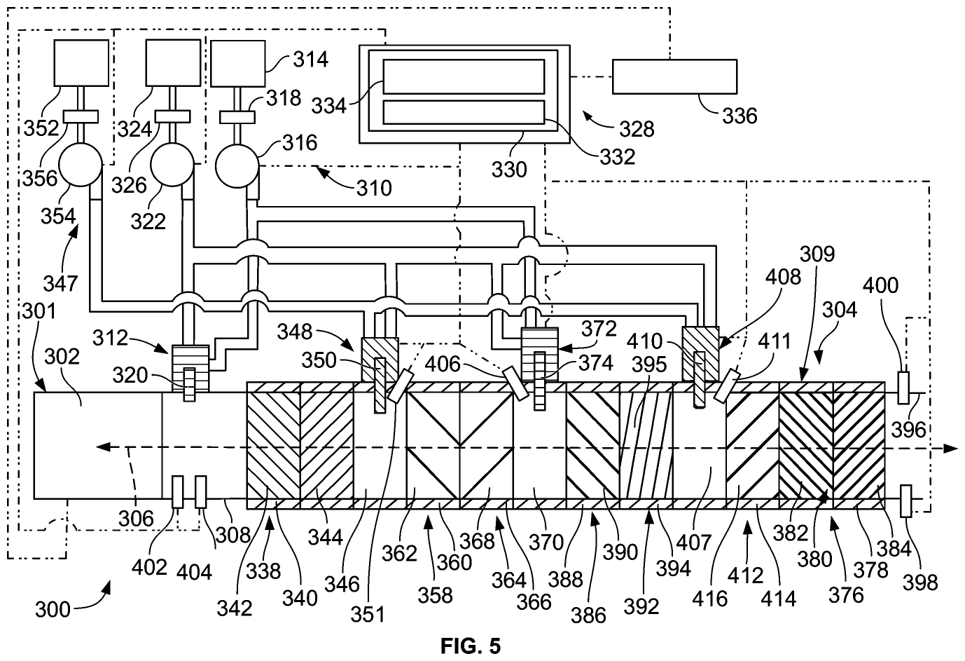

Referring to FIGS. 3-5, an aftertreatment system 300 (e.g., treatment system, etc.) for an internal combustion engine system 301. The internal combustion engine system 301 includes an internal combustion engine (e.g., diesel internal combustion engine, gasoline internal combustion engine, hybrid internal combustion engine, propane internal combustion engine, dual-fuel internal combustion engine, etc.). The internal combustion engine system 301 includes an engine control component 302 configured to control operation of the internal combustion engine. The aftertreatment system 300 is configured to treat exhaust gas produced by the internal combustion engine. As is explained in more detail herein, the aftertreatment system 300 is configured to facilitate treatment of the exhaust gas. The treatment may facilitate reduction of emission of undesirable components (e.g., nitrogen oxides (NOₓ), sulfur oxide (SOₓ), etc.) in the exhaust gas. The treatment may also or instead facilitate conversion of various oxidation components (e.g., carbon monoxide (CO), hydrocarbons, etc.) of the exhaust gas into other components (e.g., CO₂, water vapor, etc.). The treatment may also or instead facilitate removal of particulates (e.g., soot, particulate matter, etc.) from the exhaust gas.

The aftertreatment system 300 includes an exhaust gas conduit system 304 (e.g., line system, pipe system, etc.). The exhaust gas conduit system 304 is configured to facilitate routing of the exhaust gas produced by the internal combustion engine throughout the aftertreatment system 300 and to atmosphere (e.g., ambient environment, etc.). The exhaust gas conduit system 304 is centered on a conduit center axis 306 (e.g., the conduit center axis 306 extends through a center point of the exhaust gas conduit system 304, etc.).

The exhaust gas conduit system 304 includes an intake chamber 308 (e.g., line, pipe, etc.). The intake chamber 308 is configured to receive exhaust gas from the engine control component 302. The intake chamber 308 may receive exhaust gas from a portion of the internal combustion engine (e.g., header on the internal combustion engine, exhaust manifold on the internal combustion engine, the internal combustion engine, etc.) In some embodiments, the intake chamber 308 is coupled (e.g., attached, fixed, welded, fastened, riveted, adhesively attached, bonded, pinned, press-fit, etc.) to the internal combustion engine. In other embodiments, the intake chamber 308 is integrally formed with the internal combustion engine. The intake chamber 308 may be centered on the conduit center axis 306 (e.g., the conduit center axis 306 extends through a center point of the intake chamber 308, etc.). In some embodiments, the intake chamber 308 may be offset from the conduit center axis 306 (e.g., the conduit center axis 306 extends adjacent to a center point of the intake chamber 308, etc.).

In some embodiments, the exhaust gas conduit system 304 also includes an introduction conduit 309 (e.g., decomposition housing, decomposition reactor, decomposition chamber, reactor pipe, decomposition tube, reactor tube, etc.). The introduction conduit 309 is configured to receive exhaust gas from the intake chamber 308. In various embodiments, the introduction conduit 309 is coupled to the intake chamber 308. For example, the introduction conduit 309 may be fastened (e.g., using a band, using bolts, using twist-lock fasteners, threaded, etc.), welded, riveted, or otherwise attached to the intake chamber 308. In other embodiments, the introduction conduit 309 is integrally formed with the intake chamber 308. The introduction conduit 309 is centered on the conduit center axis 306 (e.g., the conduit center axis 306 extends through a center point of the introduction conduit 309, etc.). In some embodiments, the introduction conduit 309 is formed by the coupling of the individual housings and chambers, as described herein.

The aftertreatment system 300 also includes a treatment fluid delivery system 310. As is explained in more detail herein, the treatment fluid delivery system 310 is configured to facilitate the introduction of a treatment fluid, such as a reductant (e.g., diesel exhaust fluid (DEF), Adblue®, a urea-water solution (UWS), an aqueous urea solution, AUS32, etc.) or a hydrocarbon (e.g., fuel, oil, additive, etc.), into the exhaust gas within the exhaust gas. When the reductant is introduced into the exhaust gas, reduction of emission of undesirable components in the exhaust gas using the aftertreatment system 300 may be facilitated. When the hydrocarbon is introduced into the exhaust gas, the temperature of the exhaust gas may be increased (e.g., to facilitate regeneration of components of the aftertreatment system 300, etc.). For example, the temperature of the exhaust gas may be increased by combusting the hydrocarbon within the exhaust gas (e.g., using a spark plug, etc.).

The treatment fluid delivery system 310 includes an intake chamber dosing module 312 (e.g., doser, reductant doser, etc.). The intake chamber dosing module 312 is configured to facilitate passage of the treatment fluid through the intake chamber 308 and into intake chamber 308. In some embodiments, the intake chamber dosing module 312 is positioned within a dosing module mount. The dosing module mount is configured to facilitate mounting of the intake chamber dosing module 312 to the intake chamber 308. The dosing module mount may provide insulation (e.g., thermal insulation, vibrational insulation, etc.) between the intake chamber dosing module 312 and the intake chamber 308. In some embodiments, the treatment fluid delivery system 310 does not include the intake chamber dosing module 312.

The treatment fluid delivery system 310 also includes a treatment fluid source 314 (e.g., reductant tank, etc.). The treatment fluid source 314 is configured to contain the treatment fluid. The treatment fluid source 314 is configured to provide the treatment fluid to the intake chamber dosing module 312. The treatment fluid source 314 may include multiple treatment fluid sources 314 (e.g., multiple tanks connected in series or in parallel, etc.). The treatment fluid source 314 may be, for example, a diesel exhaust fluid tank containing Adblue®.

The treatment fluid delivery system 310 also includes a treatment fluid pump 316 (e.g., supply unit, etc.). The treatment fluid pump 316 is configured to receive the treatment fluid from the treatment fluid source 314 and to provide the treatment fluid to the intake chamber dosing module 312. The treatment fluid pump 316 is used to pressurize the treatment fluid from the treatment fluid source 314 for delivery to the intake chamber dosing module 312. In some embodiments, the treatment fluid pump 316 is pressure controlled. In some embodiments, the treatment fluid pump 316 is coupled to a chassis of a vehicle associated with the aftertreatment system 300.

In some embodiments, the treatment fluid delivery system 310 also includes a treatment fluid filter 318. The treatment fluid filter 318 is configured to receive the treatment fluid from the treatment fluid source 314 and to provide the treatment fluid to the treatment fluid pump 316. The treatment fluid filter 318 filters the treatment fluid prior to the treatment fluid being provided to internal components of the treatment fluid pump 316. For example, the treatment fluid filter 318 may inhibit or prevent the transmission of solids to the internal components of the treatment fluid pump 316. In this way, the treatment fluid filter 318 may facilitate prolonged desirable operation of the treatment fluid pump 316.

The intake chamber dosing module 312 includes at least one intake chamber dosing module injector 320 (e.g., insertion device, etc.). The intake chamber dosing module injector 320 is configured to receive the treatment fluid from the treatment fluid pump 316. The intake chamber dosing module injector 320 is configured to dose (e.g., provide, inject, insert, etc.) the treatment fluid received by the intake chamber dosing module 312 into the exhaust gas within the intake chamber 308.

In some embodiments, the treatment fluid delivery system 310 also includes an air pump 322 and an air source 324 (e.g., air intake, etc.). The air pump 322 is configured to receive air from the air source 324. The air pump 322 is configured to provide the air to the intake chamber dosing module 312. In some applications, the intake chamber dosing module 312 is configured to mix the air and the treatment fluid into an air-treatment fluid mixture and to provide the air-treatment fluid mixture to the intake chamber dosing module injector 320 (e.g., for dosing into the exhaust gas within the intake chamber 308, etc.). As used herein, it is understood that a treatment fluid may include or consistent of an air-treatment fluid mixture.

The intake chamber dosing module injector 320 is configured to receive the air from the air pump 322. The intake chamber dosing module injector 320 is configured to dose the air into the exhaust gas within the intake chamber 308. In some of these embodiments, the treatment fluid delivery system 310 also includes an air filter 326. The air filter 326 is configured to receive the air from the air source 324 and to provide the air to the air pump 322. The air filter 326 is configured to filter the air prior to the air being provided to the air pump 322. In other embodiments, the treatment fluid delivery system 310 does not include the air pump 322 and/or the treatment fluid delivery system 310 does not include the air source 324. In such embodiments, the intake chamber dosing module 312 is not configured to mix the treatment fluid with the air.

In various embodiments, the intake chamber dosing module 312 is configured to receive air and fluid, and doses the treatment fluid into the intake chamber 308. In various embodiments, the intake chamber dosing module 312 is configured to receive treatment fluid (and does not receive air), and doses the treatment fluid into the intake chamber 308. In various embodiments, the intake chamber dosing module 312 is configured to receive treatment fluid, and doses the treatment fluid into the intake chamber 308. In various embodiments, the intake chamber dosing module 312 is configured to receive air and treatment fluid, and doses the treatment fluid into the intake chamber 308.

The aftertreatment system 300 also includes an aftertreatment system controller 328 (e.g., control circuit, driver, etc.). The intake chamber dosing module 312, the treatment fluid pump 316, and the air pump 322 are also electrically or communicatively coupled to the aftertreatment system controller 328. The aftertreatment system controller 328 is configured to control the intake chamber dosing module 312 to dose the treatment fluid into the intake chamber 308. The aftertreatment system controller 328 may also be configured to control the treatment fluid pump 316 and/or the air pump 322 in order to control the treatment fluid that is dosed into the intake chamber 308.

The aftertreatment system controller 328 includes an aftertreatment system processing circuit 330. The aftertreatment system processing circuit 330 includes an aftertreatment system processor 332 and an aftertreatment system memory 334. The aftertreatment system processor 332 may include a microprocessor, an application-specific integrated circuit (ASIC), a field-programmable gate array (FPGA), etc., or combinations thereof. The aftertreatment system memory 334 may include, but is not limited to, electronic, optical, magnetic, or any other storage or transmission device capable of providing a processor, ASIC, FPGA, etc. with program instructions. The aftertreatment system memory 334 may include a memory chip, Electrically Erasable Programmable Read-Only Memory (EEPROM), Erasable Programmable Read Only Memory (EPROM), flash memory, or any other suitable memory from which the aftertreatment system controller 328 can read instructions. The instructions may include code from any suitable programming language. The aftertreatment system memory 334 may include various modules that include instructions that are configured to be implemented by the aftertreatment system processor 332.

In various embodiments, the aftertreatment system controller 328 is configured to communicate with a central controller 336 (e.g., engine control unit (ECU), engine control module (ECM), etc.) to control the engine control component 302. The engine control component 302 is configured to control the operation of a component of the internal combustion engine (e.g., throttle valve, fuel valve, air valve, ignition timing circuit, etc.) In some embodiments, the central controller 336 and the aftertreatment system controller 328 are integrated into a single controller.

In some embodiments, the central controller 336 is communicable with a display device (e.g., screen, monitor, touch screen, heads up display (HUD), indicator light, etc.). The display device may be configured to change state in response to receiving information from the central controller 336. For example, the display device may be configured to change between a static state and an alarm state based on a communication from the central controller 336. By changing state, the display device may provide an indication to a user of a status of the treatment fluid delivery system 310.

The aftertreatment system 300 includes an upstream catalyst member 338 (e.g., conversion catalyst member, SCR catalyst member, catalyst metals, etc.). The upstream catalyst member 338 is positioned downstream of the intake chamber 308. The upstream catalyst member 338 is configured to cause decomposition of components of the exhaust gas using the treatment fluid (e.g., via catalytic reactions, etc.). The upstream catalyst member 338 includes an upstream catalyst housing 340. The upstream catalyst housing 340 may be coupled to the intake chamber 308. In some embodiments, the upstream catalyst housing 340 is integrally formed with the intake chamber 308. The upstream catalyst member 338 includes an upstream catalyst substrate 342. The upstream catalyst substrate 342 is coupled to the upstream catalyst housing 340. In some embodiments, the upstream catalyst substrate 342 is integrally formed with the upstream catalyst housing 340.

The upstream catalyst member 338 receives the exhaust gas from the intake chamber 308. The exhaust gas flows through the upstream catalyst substrate 342 and reacts with the upstream catalyst substrate 342 so as to cause the exhaust gas to undergo the processes of evaporation, thermolysis, and/or hydrolysis to form non-NOₓ emissions within the introduction conduit 309 and/or the upstream catalyst member 338. In some embodiments, the exhaust gas and the treatment fluid within the exhaust gas react with the upstream catalyst substrate 342. In this way the upstream catalyst member 338 is configured to assist the reduction of NOₓ emissions by accelerating a NOₓ reduction process between the reductant and the NOₓ of the exhaust gas into diatomic nitrogen, water, and/or carbon dioxide and also configured to assist in the reduction of particulates from the exhaust gas. The upstream catalyst substrate 342 may include vanadia. Vanadia may be used due to the lengthy deactivation time and the ability to react with the exhaust gas at high temperatures. In some embodiments, vanadia is used because of the benefit of emitting lower N₂O emissions into the environment when exhaust gas temperatures are below 420° C. In some embodiments, the aftertreatment system 300 does not include an upstream catalyst member 338.

The aftertreatment system 300 includes an upstream ammonia slip catalyst substrate 344. The upstream ammonia slip catalyst substrate 344. The upstream ammonia slip catalyst substrate 344 is positioned downstream of the upstream catalyst member 338. In some embodiments, the upstream ammonia slip catalyst substrate 344 is a coating applied to a portion of the outlet of the upstream catalyst member 338. The upstream ammonia slip catalyst substrate 344 is configured to receive the exhaust gas from the upstream catalyst member 338 and assist in the reduction of the byproducts (e.g., ammonia, etc.) of the processes of the intake chamber dosing module 312 and the upstream catalyst member 338. Specifically, the intake chamber dosing module 312 may introduce ammonia into the exhaust gas, however a portion of the ammonia introduced may not react with the exhaust gas. As a result, the excess ammonia may slip the upstream catalyst member 338 into the exhaust gas downstream of the upstream catalyst member 338 such that the exhaust gas downstream of the upstream ammonia slip catalyst substrate 344 does not contain an undesirable amount of ammonia. In some embodiments, the aftertreatment system 300 does not include the upstream ammonia slip catalyst substrate 344.

The aftertreatment system 300 also includes a first hydrocarbon decomposition chamber 346. The first hydrocarbon decomposition chamber 346 is positioned downstream of the upstream ammonia slip catalyst substrate 344. In some embodiments the first hydrocarbon decomposition chamber 346 is coupled to the upstream catalyst housing 340. In some embodiments, the hydrocarbon decomposition chamber is integrally formed with the upstream catalyst housing 340. In still other embodiments, the first hydrocarbon decomposition chamber 346 is coupled to the intake chamber 308. In some embodiments, the aftertreatment system 300 does not include the upstream catalyst member 338 such that the first hydrocarbon decomposition chamber 346 may also be integrally formed with the intake chamber 308. The first hydrocarbon decomposition chamber 346 is configured to receive the exhaust gas from the upstream ammonia slip catalyst substrate 344. The aftertreatment system 300 includes a hydrocarbon fluid system 347. The hydrocarbon fluid system 347 includes a first hydrocarbon dosing module 348. The first hydrocarbon dosing module 348 doses the exhaust gas within the first hydrocarbon decomposition chamber 346 with hydrocarbons. The first hydrocarbon dosing module 348 is configured to facilitate passage of hydrocarbon through the first hydrocarbon decomposition chamber 346 and into the first hydrocarbon decomposition chamber 346. The first hydrocarbon dosing module 348 includes at least one first hydrocarbon injector 350 (e.g., insertion device, etc.). The first hydrocarbon injector 350 is configured to dose the hydrocarbons into the exhaust gas within the first hydrocarbon decomposition chamber 346.

The hydrocarbons within the first hydrocarbon decomposition chamber 346 may be configured to increase the temperature of the exhaust gas within the first hydrocarbon decomposition chamber 346. Specifically, the aftertreatment system 300 includes a first igniter 351 (e.g., spark plug, etc.) coupled to the first hydrocarbon decomposition chamber 346. The first igniter 351 is electrically connected to the aftertreatment system controller 328 and is configured to combust the hydrocarbons within the first hydrocarbon decomposition chamber 346 which causes the increase in temperature of the exhaust gas. By this way, regeneration of downstream components may occur.

The hydrocarbon fluid system 347 further includes a hydrocarbon source 352 (e.g., hydrocarbon tank, etc.). The hydrocarbon source 352 is configured to contain hydrocarbon. The hydrocarbon source 352 is configured to provide hydrocarbons to the first hydrocarbon dosing module 348. The hydrocarbon source 352 may include multiple hydrocarbon sources 352 (e.g., multiple tanks connected in series or in parallel, etc.). The treatment fluid delivery system also includes a hydrocarbon pump 354. Specifically, the hydrocarbon pump 354 is configured to provide hydrocarbons to the first hydrocarbon injector 350. The first hydrocarbon injector 350 receives hydrocarbon from the hydrocarbon pump 354 and is configured to dose the hydrocarbon received by the first hydrocarbon dosing module 348 into the exhaust gas within the first hydrocarbon decomposition chamber 346. The hydrocarbon pump 354 is used to pressurize hydrocarbons from the hydrocarbon source 352 for delivery to the first hydrocarbon dosing module 348 and the first hydrocarbon injector 350. In some embodiments, the hydrocarbon pump 354 is pressure controlled. In some embodiments the hydrocarbon pump 354 is coupled to a chassis of a vehicle associated with the aftertreatment system.

In some embodiments, the hydrocarbon fluid system 547 includes a hydrocarbon filter 356 (e.g., fuel filter, lubricant filter, oil filter, etc.). The hydrocarbon filter 356 is configured to receive the hydrocarbons from the hydrocarbon source 352 and to provide the hydrocarbons to the hydrocarbon pump 354. The hydrocarbon filter 356 filters the hydrocarbons prior to the hydrocarbons being provided to internal components of the hydrocarbon pump 354. For example, the hydrocarbon filter 356 may inhibit or prevent the transmission of solids to the internal components of the hydrocarbon pump 354. In this way, the hydrocarbon filter 356 may facilitate prolonged desirable operation of the hydrocarbon pump 354.

In some embodiments, the air pump 322 is also configured to provide the air to the first hydrocarbon dosing module 348. The first hydrocarbon dosing module 348 is configured to provide the air into the first hydrocarbon decomposition chamber 346. In some applications, the first hydrocarbon dosing module 348 is configured to mix the air and the hydrocarbons into an air-hydrocarbon fluid mixture and to provide the air-hydrocarbon fluid mixture to the first hydrocarbon injector 350 (e.g., for dosing into the exhaust gas within the first hydrocarbon decomposition chamber 346, etc.).

In various embodiments, the first hydrocarbon dosing module 348 is configured to receive air and hydrocarbon, and doses the hydrocarbons into the first hydrocarbon decomposition chamber 346. In various embodiments, the first hydrocarbon dosing module 348 is configured to receive hydrocarbons (and does not receive air), and doses the hydrocarbon into the intake chamber 308. In various embodiments, the first hydrocarbon dosing module 348 is configured to receive hydrocarbons, and doses the hydrocarbon into the first hydrocarbon decomposition chamber 346.

In some embodiments, the first hydrocarbon dosing module 348 and the hydrocarbon pump 354 are also electrically or communicatively coupled to the aftertreatment system controller 328. The aftertreatment system controller 328 is further configured to control the first hydrocarbon dosing module 348 to dose the hydrocarbon into the first hydrocarbon decomposition chamber 346. The aftertreatment system controller 328 may also be configured to control the hydrocarbon pump 354 and/or the air pump 322 in order to control the hydrocarbon that is dosed into the first hydrocarbon decomposition chamber 346. In some embodiments, the aftertreatment system 300 does not include the first hydrocarbon decomposition chamber 346, the first hydrocarbon dosing module 348, the first hydrocarbon injector 350, the hydrocarbon source 352, the hydrocarbon pump 354, and/or the hydrocarbon filter 356.

The aftertreatment system 300 includes a first oxidation catalyst member 358 (e.g., first DOC, etc.). The first oxidation catalyst member 358 is positioned downstream of first hydrocarbon decomposition chamber 346 (i.e., the first hydrocarbon decomposition chamber 346 is positioned upstream of the first oxidation catalyst member 358).

The first oxidation catalyst member includes a first oxidation catalyst housing 360. The first oxidation catalyst housing 360 is coupled to first hydrocarbon decomposition chamber 346. The first oxidation catalyst housing 360 may also be integrally formed with the first hydrocarbon decomposition chamber 346.

The first oxidation catalyst member 358 also includes a first oxidation catalyst substrate 362 (e.g., DOC, etc.). The first oxidation catalyst substrate 362 is positioned within the first oxidation catalyst housing 360. The first oxidation catalyst substrate 362 may be coupled to the first oxidation catalyst housing 360. The exhaust gas including hydrocarbons react with the first oxidation catalyst substrate 362 and cause the conversion of the hydrocarbons in the exhaust gas. For example, as the exhaust gas flows the through the first oxidation catalyst substrate 362, the hydrocarbons react with the first oxidation catalyst substrate 362 and began to oxidize. The first oxidation catalyst substrate 362 facilitates conversion of the carbon monoxide in the exhaust gas and the hydrocarbons and/or the air-hydrocarbon mixture into carbon dioxide.

In some embodiments, the aftertreatment system 300 does not include the upstream catalyst member 338, the upstream ammonia slip catalyst substrate 344, and/or the first hydrocarbon decomposition chamber 346 such that the first oxidation catalyst member 358 may be positioned downstream of the intake chamber 308. In such embodiments, the first oxidation catalyst housing 360 may be coupled to the intake chamber 308. In other such embodiments, the first oxidation catalyst housing 360 may also be integrally formed with the intake chamber 308.

The aftertreatment system 300 also includes an upstream particulate filter assembly 364. The upstream particulate filter assembly 364 includes an upstream particulate filter housing 366. The upstream particulate filter housing 366 is positioned downstream of the first oxidation catalyst housing 360. In some embodiments, the upstream particulate filter housing 366 is integrally formed with the first oxidation catalyst housing 360. The upstream particulate filter assembly 364 also includes an upstream particulate filter 368 (e.g., DPF, filtration member, etc.). The upstream particulate filter 368 is disposed within the upstream particulate filter housing 366 such that the upstream particulate filter 368 is positioned downstream of the first oxidation catalyst member 358 (i.e., the first oxidation catalyst member 358 is positioned upstream of the upstream particulate filter 368). In some embodiments, the upstream particulate filter housing 366 and the upstream particulate filter 368 are positioned downstream of the intake chamber 308.

The upstream particulate filter 368 is configured to remove first particulates (e.g., soot, solidified hydrocarbons, ash, etc.) from the exhaust gas. For example, the upstream particulate filter 368 may receive exhaust gas (e.g., from the first oxidation catalyst member 358, the intake chamber 308 etc.) having a first concentration of the first particulates and may provide the exhaust gas downstream having a second concentration of the first particulates, where the second concentration is lower than the first concentration. In this way, the upstream particulate filter 368 may facilitate reduction of a particulate number (PN) of the exhaust gas. Decreasing the PN of the exhaust gas may be desirable in a variety of applications. For example, emissions regulations may prescribe a maximum PN for exhaust gas emitted to atmosphere and the upstream particulate filter 368 may ensure that the PN of the exhaust gas emitted to atmosphere by the aftertreatment system 300 is below the maximum PN.

In some embodiments, the upstream particulate filter 368 is a catalyzed DPF. The catalyzed DPF is a filter that has a catalyst coating. The catalyst coating is configured to react with a component of the exhaust gas to reduce undesirable components in the exhaust. For example, the catalyst coating could be an oxidation catalyst to reduce hydrocarbons within the exhaust gas. In some embodiments, the catalyst coating is a SCR catalyst configured to reduce NOₓ emissions.

The aftertreatment system 300 includes a decomposition chamber 370 (e.g., decomposition reactor, decomposition reactor tube (DRT), etc.). The decomposition chamber 370 is positioned downstream of the upstream particulate filter assembly 364 (i.e., the decomposition chamber 370 is positioned downstream of the upstream particulate filter 368) and configured to receive exhaust gas from the upstream particulate filter assembly 364. The decomposition chamber 370 may be coupled to the upstream particulate filter housing 366. In some embodiments, the decomposition chamber 370 is integrally formed with the upstream particulate filter housing 366.

The treatment fluid delivery system 310 includes a decomposition chamber dosing module 372. The decomposition chamber dosing module 372 is configured to facilitate passage of the treatment fluid through the decomposition chamber 370 and into the decomposition chamber 370.

In some embodiments, the decomposition chamber dosing module 372 is positioned within a dosing module mount. The dosing module mount is configure to facilitate mounting of the decomposition chamber dosing module 372 to the decomposition chamber 370. The dosing module mount may provide insulation (e.g., thermal insulation, vibrational insulation, etc.) between the decomposition chamber dosing module 372 and the decomposition chamber 370.

The decomposition chamber dosing module 372 includes at least one decomposition chamber injector 374 (e.g., insertion device, etc.). The decomposition chamber injector 374 is configured to receive the treatment fluid from the treatment fluid pump 316. The decomposition chamber injector 374 is configured to dose the treatment fluid received by the decomposition chamber dosing module 372 into the exhaust gas within the decomposition chamber 370. When the treatment fluid is introduced into the exhaust gas, reduction of emission of undesirable particulates in the exhaust gas may be facilitated.

In some embodiments, the decomposition chamber dosing module 372 is configured to receive air from the air pump 322. In some applications, the decomposition chamber dosing module 372 is configured to mix the air and the treatment fluid into an air-treatment fluid mixture and to provide the air-treatment fluid mixture to the decomposition chamber injector 374 (e.g., for dosing into the exhaust gas within the decomposition chamber 370, etc.). Specifically, the decomposition chamber injector 374 is configured to receive the air from the air pump 322. The intake chamber dosing module injector 320 is configured to dose the air into the exhaust gas within the decomposition chamber 370.

In various embodiments, the decomposition chamber dosing module 372 is configured to receive air and fluid, and doses the treatment fluid into the decomposition chamber 370. In various embodiments, the decomposition chamber dosing module 372 is configured to receive treatment fluid (and does not receive air), and doses the treatment fluid into the decomposition chamber 370. In various embodiments, the decomposition chamber dosing module 372 is configured to receive treatment fluid, and doses the treatment fluid into the decomposition chamber 370. In various embodiments, the decomposition chamber dosing module 372 is configured to receive air and treatment fluid, and doses the treatment fluid into the decomposition chamber 370.

The aftertreatment system 300 includes a downstream particulate filter assembly 376. The downstream particulate filter assembly 376 includes a downstream particulate filter housing 378 and a downstream particulate filter 380 (e.g. DPF, filtration member, catalyzed filter member, selective catalyst reduction filter, etc.). The downstream particulate filter housing 378 is positioned downstream of the decomposition chamber 370. In some embodiments, the downstream particulate filter housing 378 is integrally formed with the decomposition chamber 370. The downstream particulate filter 380 may be a catalyzed filter member and is disposed within the downstream particulate filter housing 378 such that the downstream particulate filter 380 is positioned downstream of the decomposition chamber 370. The downstream particulate filter 380 is configured to remove second particulates (e.g., soot, solidified hydrocarbons, ash, etc.) from the exhaust gas.

The downstream particulate filter 380 includes a filter substrate. The filter substrate of the downstream particulate filter 380 is configured to filter second particulates from the exhaust gas. The filter substrate of the downstream particulate filter 380 includes a filter substrate inlet 382 and a filter substrate outlet 384. The filter substrate inlet 382 is configured to contact the exhaust gas received by the filter substrate and capture the second particulates in the exhaust gas. The filter substrate inlet 382 facilitates the exhaust gas to flow toward the filter substrate outlet 384. The filter substrate outlet 384 is configured to contact the exhaust gas provided by the filter substrate and facilitate the exhaust gas downstream.

The filter substrate of the downstream particulate filter 380 includes a catalyst coating. The catalyst coating may include an inlet coating which is applied to at least a portion of the surface of the filter substrate inlet 382. The catalyst coating may include an outlet coating which is applied to at least a portion of a surface of the filter substrate outlet 384. The inlet coating comprises an iron zeolite catalyst coating, a copper zeolite catalyst coating, or an ammonia slip catalyst coating. The outlet coating comprises an iron zeolite catalyst coating, a copper zeolite catalyst coating, or an ammonia slip catalyst coating. In some embodiments the inlet coating is iron zeolite. The outlet coating may be copper zeolite. By coating the filter substrate inlet 382 and/or the filter substrate outlet 384 of the filter substrate of the downstream particulate filter 380, the exhaust gas is caused to react with the inlet coating and the outlet coating and undergoes the process of processes of evaporation, thermolysis, and/or hydrolysis to form non-NOₓ emissions within the introduction conduit 309 and/or the downstream particulate filter assembly 376. In this way, the downstream particulate filter 380 is configured to assist the reduction of NOₓ of the exhaust gas into diatomic nitrogen, water, and/or carbon dioxide and also configured to assist in the reduction of particulates from the exhaust gas.

The aftertreatment system 300 includes a first downstream catalyst member 386 (e.g., conversion catalyst member, SCR catalyst member, catalyst metals, etc.). The first downstream catalyst member 386 is positioned downstream of the downstream particulate filter assembly 376, and therefore, the first downstream catalyst member 386 is positioned downstream of downstream particulate filter 380. The first downstream catalyst member 386 is configured to cause decomposition of components of the exhaust gas using the treatment fluid (e.g., via catalytic reactions, etc.). The first downstream catalyst member 386 includes a first downstream catalyst housing 388 and a first downstream catalyst substrate 390. The first downstream catalyst housing 388 may be coupled to the downstream particulate filter housing 378. In some embodiments, the first downstream catalyst housing 388 is integrally formed with the downstream particulate filter housing 378. The first downstream catalyst substrate 390 is coupled to the first downstream catalyst housing 388. In some embodiments, the first downstream catalyst substrate 390 is integrally formed with the first downstream catalyst housing 388.

The first downstream catalyst member 386 receives the exhaust gas from the downstream particulate filter assembly 376. The exhaust gas flows through the first downstream catalyst substrate 390 and reacts with the first downstream catalyst substrate 390 so as to cause the exhaust gas to undergo the processes of evaporation, thermolysis, and/or hydrolysis to form non-NOₓ emissions within the introduction conduit 309 and/or the first downstream catalyst member 386. In some embodiments, the exhaust gas and the treatment fluid within the exhaust gas react with the first downstream catalyst substrate 390. In this way the first downstream catalyst member 386 is configured to assist the reduction of NOₓ emissions by accelerating a NOₓ reduction process between the reductant and the NOₓ of the exhaust gas into diatomic nitrogen, water, and/or carbon dioxide and also configured to assist in the reduction of particulates from the exhaust gas. The first downstream catalyst member 386 may include iron zeolite. The first downstream catalyst member 386 may include copper zeolite. In some embodiments, the aftertreatment system 300 does not include a first downstream catalyst member 386.

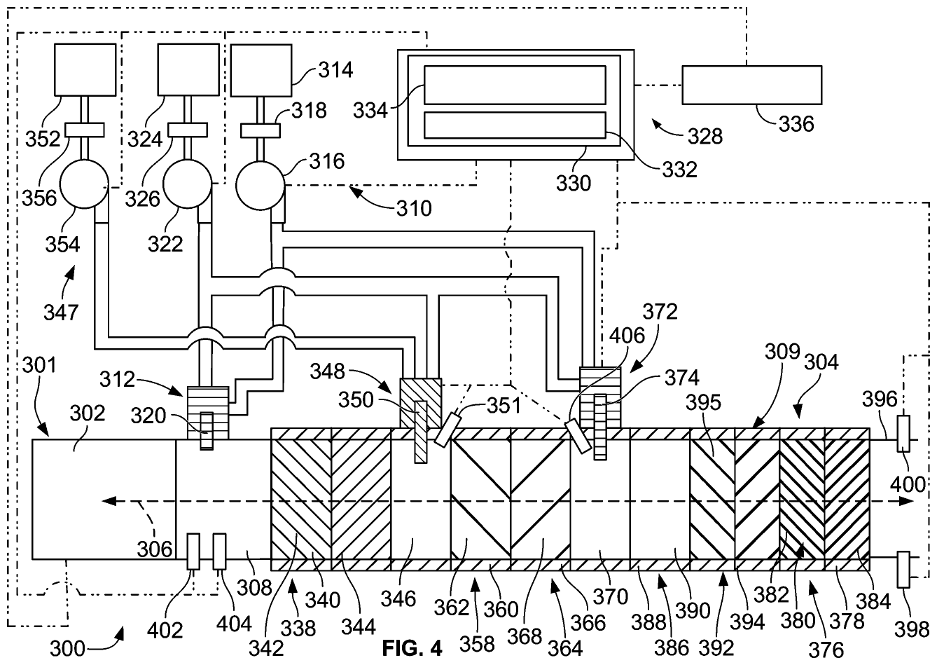

As seen FIG. 4, in some embodiments, the first downstream catalyst member 386, including the first downstream catalyst housing 388 and the first downstream catalyst substrate 390, is positioned downstream of the decomposition chamber 370 and upstream of the downstream particulate filter assembly 376 (e.g., upstream of the downstream particulate filter 380, etc.). The first downstream catalyst housing 388 may be coupled to the decomposition chamber 370. In some embodiments, the first downstream catalyst housing 388 may be integrally formed with the decomposition chamber 370.

Referring back to FIG. 3, the aftertreatment system 300 includes a second downstream catalyst member 392 (e.g., conversion catalyst member, SCR catalyst member, catalyst metals, etc.). The second downstream catalyst member 392 is positioned downstream of the first downstream catalyst member 386. The second downstream catalyst member 392 includes a second downstream catalyst housing 394. In some embodiments, the second downstream catalyst housing 394 is integrally formed with the first downstream catalyst housing 388. In some embodiments, the second downstream catalyst housing 394 is the first downstream catalyst housing 388. The second downstream catalyst member 392 also includes a second downstream catalyst substrate 395. The second downstream catalyst substrate 395 is coupled to the second downstream catalyst housing 394. In some embodiments, the second downstream catalyst substrate 395 is integrally formed with the second downstream catalyst housing 394. In some embodiments, the second downstream catalyst member 392 does not include a second downstream catalyst housing 394 and is a coating applied to a portion of the outlet of the first downstream catalyst member 386. In some embodiments, the aftertreatment system 300 does not include a second downstream catalyst member 392.

In some embodiments, the second downstream catalyst substrate 395 is an ammonia slip catalyst substrate configured to assist in the reduction of the byproducts (e.g., ammonia, etc.) of the process of the decomposition chamber dosing module 372 and the first downstream catalyst member 386. The second downstream catalyst member 392 receives the exhaust gas from the first downstream catalyst member 386. The exhaust gas flows through the second downstream catalyst substrate 395. The second downstream catalyst substrate 395 is configured to react with the exhaust gas and the treatment fluid within the exhaust gas provided by first downstream catalyst member 386. Specifically, the decomposition chamber dosing module 372 may introduce ammonia into the exhaust gas, however a portion of the ammonia introduced may not react with the exhaust gas. As a result, the excess ammonia may slip from the first downstream catalyst member 386 into the exhaust gas downstream of the first downstream catalyst member 386 such that the exhaust gas downstream of the downstream ammonia slip catalyst substrate 188 does not contain an undesirable amount of ammonia.

As seen in FIG. 4, in some embodiments, the second downstream catalyst member 392 is positioned downstream of the first downstream catalyst member 386. The second downstream catalyst member 392 may be configured to cause decomposition of components of the exhaust gas using the treatment fluid (e.g., via catalytic reactions, etc.). The second downstream catalyst housing 394 is coupled to the first downstream catalyst housing 388. In some embodiments, the second downstream catalyst housing 394 is integrally formed with the first downstream catalyst housing 388. In some embodiments, the second downstream catalyst housing 394 is the first downstream catalyst housing 388. The second downstream catalyst substrate 395 is coupled to the second downstream catalyst housing 394. In some embodiments, the second downstream catalyst substrate 395 is integrally formed with the second downstream catalyst housing 394.

The second downstream catalyst member 392 receives the exhaust gas from the first downstream catalyst member 386. The exhaust gas flows through the second downstream catalyst substrate 395. The second downstream catalyst substrate 395 is configured to react with the exhaust gas and the treatment fluid within the exhaust gas provided by first downstream catalyst member 386 and cause the exhaust gas to undergo the processes of evaporation, thermolysis, and/or hydrolysis to form non-NOₓ emissions within the introduction conduit 309 and/or the second downstream catalyst member 392. In this way the second downstream catalyst member 392 is configured to assist the reduction of NOₓ emissions by accelerating a NOₓ reduction process between the reductant and the NOₓ of the exhaust gas into diatomic nitrogen, water, and/or carbon dioxide and also configured to assist in the reduction of particulates from the exhaust gas. The second downstream catalyst member 392 may include iron zeolite. The second downstream catalyst member 392 may include copper zeolite.

As seen in FIG. 4, the downstream particulate filter assembly 376 may be positioned downstream of the second downstream catalyst member 392. For example, the downstream particulate filter housing 378 may be coupled to the second downstream catalyst housing 394. In some embodiments, the downstream particulate filter housing 378 is integrally formed to the second downstream catalyst housing 394. Further, the inlet coating of filter substrate inlet 382 is copper zeolite or iron zeolite and the outlet coating of the filter substrate outlet 384 is ammonia slip. In some embodiments, the inlet coating of the filter substrate inlet 382 and the outlet coating of the filter substrate outlet 384 is ammonia slip.

Referring to FIG. 3, the aftertreatment system 300 also includes an outlet chamber 396. The outlet chamber 396 is positioned downstream of the second downstream catalyst member 392 and is configured to receive the exhaust gas from the second downstream catalyst member 392. In various embodiments, the outlet chamber 396 is coupled to the second downstream catalyst housing 394. For example, the outlet chamber 396 may be fastened, welded, riveted, or otherwise attached to the second downstream catalyst housing 394. In other embodiments, the outlet chamber 396 is integrally formed with the second downstream catalyst housing 394. In some embodiments, the outlet chamber 396 is coupled to the introduction conduit 309. In some embodiments, the outlet chamber 396 is the introduction conduit 309 (e.g., only the introduction conduit is included in the exhaust gas conduit system 304 and the introduction conduit 309 functions as both the introduction conduit 309 and the outlet chamber 396). The outlet chamber 396 is centered on the conduit center axis 106 (e.g., the conduit center axis 306 extends through a center point of the outlet chamber 396, etc.).

In some embodiments, as seen in FIG. 4, the outlet chamber 396 is positioned downstream of the downstream particulate filter assembly 376 (e.g., downstream of the downstream particulate filter 380) and is configured to receive the exhaust gas from the downstream particulate filter 194. In various embodiments, the outlet chamber 396 is coupled to the downstream particulate filter housing 378. In other embodiments, the outlet chamber 396 is integrally formed with the downstream particulate filter housing 378.

In various embodiments, the exhaust gas conduit system 304 only includes a single conduit that functions as the intake chamber 308, the introduction conduit 309, and the outlet chamber 396.

In various embodiments, the aftertreatment system 300 also includes a first sensor 398 (e.g., NOₓ sensor, CO sensor, CO₂ sensor, O₂ sensor, particulate sensor, nitrogen sensor, etc.). The first sensor 398 is positioned downstream of the downstream particulate filter 380. In some embodiments, the first sensor 398 is coupled to the outlet chamber 396. The first sensor 398 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid downstream of the downstream particulate filter 380. The first sensor 398 may be configured to measure the parameter within the outlet chamber 396. In some embodiments, the parameter measured by the first sensor 398 is the particulate concentration in the exhaust gas downstream of the downstream particulate filter 380. In some embodiments, the parameter measured by the first sensor 398 is the SOₓ concentration of the exhaust gas within the outlet chamber 396. In some embodiments, the first sensor 398 measures both the particulate concentration and the SOₓ concentration.

The first sensor 398 is electrically or communicatively coupled to the aftertreatment system controller 328 and is configured to provide a first signal associated with the parameter to the aftertreatment system controller 328. The aftertreatment system controller 328 (e.g., via the aftertreatment system processing circuit 330, etc.) is configured to determine a first measurement based on the first signal. The aftertreatment system controller 328 may be configured to control the intake chamber dosing module 312, the decomposition chamber dosing module 372, the treatment fluid pump 316, and/or the air pump 322 based on the first signal. Furthermore, the aftertreatment system controller 328 may be configured to communicate the first signal to the central controller 336.

In various embodiments, the aftertreatment system 300 also includes a second sensor 400 (e.g., NOₓ sensor, CO sensor, CO₂ sensor, O₂ sensor, particulate sensor, nitrogen sensor, etc.). The second sensor 400 is positioned downstream of the downstream particulate filter 380. In some embodiments, the second sensor 400 is coupled to the outlet chamber 396 and positioned downstream of the first sensor 398. The second sensor 400 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid downstream of the downstream particulate filter 380. The second sensor 400 may be configure to measure the parameter of the exhaust gas within the outlet chamber 396. In some embodiments, the parameter measured by the second sensor 400 is the particulate concentration in the exhaust gas downstream of the downstream particulate filter 380. In some embodiments, the parameter measured by the second sensor 400 is the SOₓ concentration of the exhaust gas downstream of the downstream particulate filter 380. In some embodiments, the second sensor 400 measures both the particulate concentration and the SOₓ concentration.

The second sensor 400 is electrically or communicatively coupled to the aftertreatment system controller 328 and is configured to provide a second signal associated with the parameter to the aftertreatment system controller 328. The aftertreatment system controller 328 (e.g., via the aftertreatment system processing circuit 330, etc.) is configured to determine a second measurement based on the second signal. The aftertreatment system controller 328 may be configured to control the intake chamber dosing module 312, the decomposition chamber dosing module 372, the treatment fluid pump 316, and/or the air pump 322 based on the second signal. Furthermore, the aftertreatment system controller 328 may be configured to communicate the second signal to the central controller 336.

In various embodiments, the aftertreatment system 300 also includes a third sensor 402 (e.g., NOₓ sensor, CO sensor, CO₂ sensor, O₂ sensor, particulate sensor, nitrogen sensor, etc.). The third sensor 402 is positioned upstream of the upstream particulate filter 368. In some embodiments, the third sensor 402 is coupled to the intake chamber 308. The third sensor 402 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid upstream of the upstream particulate filter 368. The third sensor 402 may be configured to measure a parameter of the exhaust gas within the intake chamber 308. In some embodiments, the parameter measured by the first sensor 398 is the particulate concentration in the exhaust gas upstream of the upstream particulate filter 368. In some embodiments, the parameter measured by the third sensor 402 is the SOₓ concentration of the exhaust gas upstream of the upstream particulate filter 368. In some embodiments, the third sensor 402 measures both the particulate concentration and the SOₓ concentration.

The third sensor 402 is electrically or communicatively coupled to the aftertreatment system controller 328 and is configured to provide a third signal associated with the parameter to the aftertreatment system controller 328. The aftertreatment system controller 328 (e.g., via the aftertreatment system processing circuit 330, etc.) is configured to determine a third measurement based on the third signal. The aftertreatment system controller 328 may be configured to control the intake chamber dosing module 312, the decomposition chamber dosing module 372, the treatment fluid pump 316, and/or the air pump 322 based on the third signal. Furthermore, the aftertreatment system controller 328 may be configured to communicate the third signal to the central controller 336.

In various embodiments, the aftertreatment system 300 also includes a fourth sensor 404 (e.g., NOₓ sensor, CO sensor, CO₂ sensor, O₂ sensor, particulate sensor, nitrogen sensor, etc.). The fourth sensor 404 is positioned upstream of the upstream particulate filter 368. In some embodiments, the fourth sensor 404 is coupled to the intake chamber 308 and positioned downstream of the third sensor 402. The fourth sensor 404 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid upstream of the upstream particulate filter 368. The fourth sensor 404 may be configured to measure a parameter of the exhaust gas upstream of the upstream particulate filter 368. In some embodiments, the parameter measured by the fourth sensor 404 is the particulate concentration in the exhaust gas upstream of the upstream particulate filter 368. In some embodiments, the parameter measured by the fourth sensor 404 is the SOₓ concentration of the exhaust gas upstream of the upstream particulate filter 368. In some embodiments, the fourth sensor 404 measures both the particulate concentration and the SOₓ concentration.

The fourth sensor 404 is electrically or communicatively coupled to the aftertreatment system controller 328 and is configured to provide a fourth signal associated with the parameter to the aftertreatment system controller 328. The aftertreatment system controller 328 (e.g., via the aftertreatment system processing circuit 330, etc.) is configured to determine a fourth measurement based on the fourth signal. The aftertreatment system controller 328 may be configured to control the intake chamber dosing module 312, the decomposition chamber dosing module 372, the treatment fluid pump 316, and/or the air pump 322 based on the fourth signal. Furthermore, the aftertreatment system controller 328 may be configured to communicate the fourth signal to the central controller 336.

In various embodiments, the aftertreatment system 300 includes a fifth sensor 406 (e.g., NOₓ sensor, CO sensor, CO₂ sensor, O₂ sensor, particulate sensor, nitrogen sensor, etc.). The fifth sensor 406 is configured substantially similar to the fifth sensor 206 and therefore not described in further detail.

In various embodiments, the aftertreatment system 300 includes a second hydrocarbon decomposition chamber 407. The second hydrocarbon decomposition chamber 407 is positioned downstream of the decomposition chamber 370 and upstream of the downstream particulate filter assembly 376 (e.g., upstream of the downstream particulate filter 380). In other embodiments, the second hydrocarbon decomposition chamber 407 is downstream of the second downstream catalyst member 392 and upstream of the downstream particulate filter assembly 376. The second hydrocarbon decomposition chamber 407 is configured to receive exhaust gas from downstream. The hydrocarbon fluid system 347 includes a second hydrocarbon dosing module 408. The second hydrocarbon dosing module 408 is configured to facilitate passage of hydrocarbon through the second hydrocarbon decomposition chamber 407 and into the second hydrocarbon decomposition chamber 407. The second hydrocarbon dosing module 408 includes at least one first hydrocarbon injector 410 (e.g., insertion device, etc.). The first hydrocarbon injector 410 is configured to receive hydrocarbons from the hydrocarbon source 352 and to dose the hydrocarbons into the exhaust gas within the second hydrocarbon decomposition chamber 407.

The hydrocarbons within the second hydrocarbon decomposition chamber 407 may be configured to increase the temperature of the exhaust gas within the second hydrocarbon decomposition chamber 407. Specifically, the aftertreatment system 300 includes a second igniter 411 (e.g., spark plug, etc.) coupled to the second hydrocarbon decomposition chamber 407. The second igniter 411 is electrically connected to the aftertreatment system controller 328 and is configured to combust the hydrocarbons within the second hydrocarbon decomposition chamber 407 which causes the increase in temperature of the exhaust gas. By this way, regeneration of downstream components may occur.

In some embodiments, the air pump 322 is also configured to provide the air to the second hydrocarbon dosing module 408. The second hydrocarbon dosing module 408 is configured to provide the air into the second hydrocarbon decomposition chamber 407. In some applications, the second hydrocarbon dosing module 408 is configured to mix the air and the hydrocarbons into an air-hydrocarbon fluid mixture and to provide the air-hydrocarbon fluid mixture to the first hydrocarbon injector 410 (e.g., for dosing into the exhaust gas within the second hydrocarbon decomposition chamber 407, etc.).

In various embodiments, the second hydrocarbon dosing module 408 is configured to receive air and hydrocarbon, and doses the hydrocarbons into the second hydrocarbon decomposition chamber 407. In various embodiments, the second hydrocarbon dosing module 408 is configured to receive hydrocarbons (and does not receive air), and doses the hydrocarbon into the intake chamber 308. In various embodiments, the second hydrocarbon dosing module 408 is configured to receive hydrocarbons, and doses the hydrocarbon into the second hydrocarbon decomposition chamber 407.

In some embodiments, the second hydrocarbon dosing module 408 and the hydrocarbon pump 354 are also electrically or communicatively coupled to the aftertreatment system controller 328. The aftertreatment system controller 328 is further configured to control the second hydrocarbon dosing module 408 to dose the hydrocarbon into the second hydrocarbon decomposition chamber 407. The aftertreatment system controller 328 may also be configured to control the hydrocarbon pump 354 and/or the air pump 322 in order to control the hydrocarbon that is dosed into the second hydrocarbon decomposition chamber 407. In some embodiments, the aftertreatment system 300 does not include the second hydrocarbon decomposition chamber 407, the second hydrocarbon dosing module 408, and the first hydrocarbon injector 410.

The aftertreatment system 300 includes a second oxidation catalyst member 412. The second oxidation catalyst member 412 is configured to receive the exhaust from downstream. The second oxidation catalyst member 412 is positioned downstream of second hydrocarbon decomposition chamber 407 (i.e., the second hydrocarbon decomposition chamber 407 is positioned upstream of the second oxidation catalyst member 412). The second oxidation catalyst member 412 includes a second oxidation catalyst housing 414. The second oxidation catalyst housing 414 is coupled to second hydrocarbon decomposition chamber 407. The second oxidation catalyst housing 414 may also be integrally formed with second hydrocarbon decomposition chamber 407. In some embodiments, the second oxidation catalyst member 412 may be positioned downstream of the decomposition chamber 370. The second oxidation catalyst housing 414 may be coupled to the second hydrocarbon decomposition chamber 407. The second oxidation catalyst housing 414 may also be integrally formed with the second hydrocarbon decomposition chamber 407.

The second oxidation catalyst member 412 includes a second oxidation catalyst substrate 416 (e.g., DOC, etc.). The second oxidation catalyst substrate 416 is positioned within the second oxidation catalyst housing 414. The second oxidation catalyst substrate 416 may be coupled to the second oxidation catalyst housing 414. The exhaust gas including hydrocarbon react with the second oxidation catalyst substrate 416 and cause the conversion of the hydrocarbons. For example, as the exhaust gas flows the through the second oxidation catalyst substrate 416, the hydrocarbons react with the second oxidation catalyst substrate 416 and began to oxidize. The second oxidation catalyst substrate 416 facilitates conversion of the carbon monoxide in the exhaust gas and the treatment fluid and/or the air-treatment fluid mixture into carbon dioxide. In some embodiments, the aftertreatment system 300 includes a second decomposition chamber positioned downstream of the second hydrocarbon decomposition chamber 407. The second decomposition chamber is substantially similar to the decomposition chamber 370. The second decomposition chamber includes a second decomposition chamber dosing module and a second decomposition chamber injector, the second decomposition chamber dosing module and the second decomposition chamber injector is substantially similar to the decomposition chamber dosing module 372 and the decomposition chamber injector 374, respectfully.

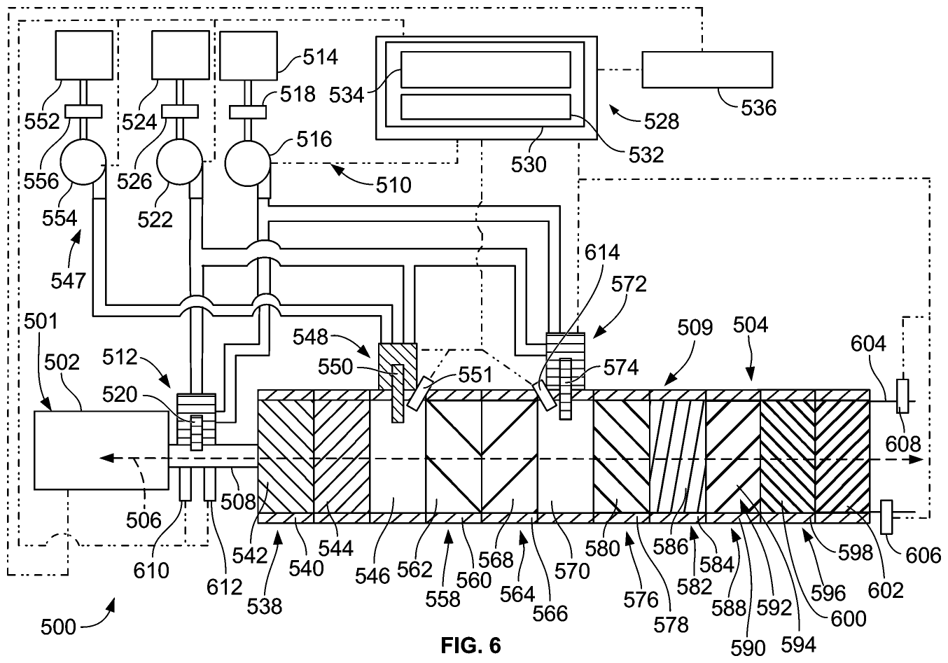

FIG. 6 depicts an aftertreatment system 500 (e.g., treatment system, etc.) for an internal combustion engine system 501 (e.g., diesel internal combustion engine, gasoline internal combustion engine, hybrid internal combustion engine, propane internal combustion engine, dual-fuel internal combustion engine, etc.). The internal combustion engine system 501 includes an engine control component 502. The aftertreatment system 500 treating exhaust gas produced by an internal combustion engine. As is explained in more detail herein, the aftertreatment system 500 is configured to facilitate treatment of the exhaust gas. The treatment may facilitate reduction of emission of undesirable components (e.g., nitrogen oxides (NOₓ), Sulfur Oxide (SOₓ), etc.) in the exhaust gas. The treatment may also or instead facilitate conversion of various oxidation components (e.g., carbon monoxide (CO), hydrocarbons, etc.) of the exhaust gas into other components (e.g., CO₂, water vapor, etc.). The treatment may also or instead facilitate removal of particulates (e.g., soot, particulate matter, etc.) from the exhaust gas.

The aftertreatment system 500 includes an exhaust gas conduit system 504 (e.g., line system, pipe system, etc.). The exhaust gas conduit system 504 is configured to facilitate routing of the exhaust gas produced by the internal combustion engine throughout the aftertreatment system 500 and to atmosphere (e.g., ambient environment, etc.). The exhaust gas conduit system 504 is centered on a conduit center axis 506 (e.g., the conduit center axis 506 extends through a center point of the exhaust gas conduit system 504 504, etc.). As used herein, the term “axis” describes a theoretical line extending through the centroid (e.g., center of mass, etc.) of an object. The object is centered on the axis. The object is not necessarily cylindrical (e.g., a non-cylindrical shape may be centered on an axis, etc.).

The exhaust gas conduit system 504 includes an intake chamber 508 (e.g., line, pipe, etc.). The intake chamber 508 is configured to receive exhaust gas from the internal combustion engine. The intake chamber may be configured to receive exhaust gas from a portion of the internal combustion engine (e.g., header on the internal combustion engine, exhaust manifold on the internal combustion engine, the internal combustion engine, etc.). In some embodiments, the intake chamber 508 is coupled (e.g., attached, fixed, welded, fastened, riveted, adhesively attached, bonded, pinned, press-fit, etc.) to the internal combustion engine. In other embodiments, the intake chamber 508 is integrally formed with the internal combustion engine. The intake chamber 508 may be centered on the conduit center axis 506 (e.g., the conduit center axis 506 extends through a center point of the intake chamber 508, etc.). In some embodiments, the intake chamber 508 may be offset from the conduit center axis 506 (e.g., the conduit center axis 506 extends adjacent to a center point of the intake chamber 508, etc.).

In some embodiments, the exhaust gas conduit system 504 also includes an introduction conduit 509 (e.g., decomposition housing, decomposition reactor, decomposition chamber, reactor pipe, decomposition tube, reactor tube, etc.). The introduction conduit 509 is configured to receive exhaust gas from the intake chamber 508. In various embodiments, the introduction conduit 509 is coupled to the intake chamber 508. For example, the introduction conduit 509 may be fastened (e.g., using a band, using bolts, using twist-lock fasteners, threaded, etc.), welded, riveted, or otherwise attached to the intake chamber 508. In other embodiments, the introduction conduit 509 is integrally formed with the intake chamber 508. As utilized herein, the terms “fastened,” “fastening,” and the like, describe attachment (e.g., joining, etc.) of two structures in such a way that detachment (e.g., separation, etc.) of the two structures remains possible while “fastened” or after the “fastening” is completed, without destroying or damaging either or both of the two structures. The introduction conduit 509 is centered on the conduit center axis 506 (e.g., the conduit center axis 506 extends through a center point of the introduction conduit 509, etc.). In some embodiments, the introduction conduit 509 is formed by the coupling of the individual housings and chambers, as described herein.

The aftertreatment system 500 also includes a treatment fluid delivery system 510. As is explained in more detail herein, the treatment fluid delivery system 510 is configured to facilitate the introduction of a treatment fluid, such as a reductant (e.g., diesel exhaust fluid (DEF), Adblue®, a urea-water solution (UWS), an aqueous urea solution, AUS32, etc.) or a hydrocarbon (e.g., fuel, oil, additive, etc.), into the exhaust gas within the exhaust gas. When the reductant is introduced into the exhaust gas, reduction of emission of undesirable components in the exhaust gas using the aftertreatment system 500 may be facilitated. When the hydrocarbon is introduced into the exhaust gas, the temperature of the exhaust gas may be increased (e.g., to facilitate regeneration of components of the aftertreatment system 500, etc.). For example, the temperature of the exhaust gas may be increased by combusting the hydrocarbon within the exhaust gas (e.g., using a spark plug, etc.).

The treatment fluid delivery system 510 includes an intake chamber dosing module 512 (e.g., doser, reductant doser, etc.). The intake chamber dosing module 512 is configured to facilitate passage of the treatment fluid through the intake chamber 508 and into intake chamber 508. In some embodiments, the intake chamber dosing module 512 is positioned within a dosing module mount. The dosing module mount is configured to facilitate mounting of the intake chamber dosing module 512 to the intake chamber 508. The dosing module mount may provide insulation (e.g., thermal insulation, vibrational insulation, etc.) between the intake chamber dosing module 512 and the intake chamber 508. In some embodiments, the treatment fluid delivery system 510 does not include the intake chamber dosing module 512.

The treatment fluid delivery system 510 also includes a treatment fluid source 514 (e.g., reductant tank, etc.). The treatment fluid source 514 is configured to contain the treatment fluid. The treatment fluid source 514 is configured to provide the treatment fluid to the intake chamber dosing module 512. The treatment fluid source 514 may include multiple treatment fluid sources 514 (e.g., multiple tanks connected in series or in parallel, etc.). The treatment fluid source 514 may be, for example, a diesel exhaust fluid tank containing Adblue®.

The treatment fluid delivery system 510 also includes a treatment fluid pump 516 (e.g., supply unit, etc.). The treatment fluid pump 516 is configured to receive the treatment fluid from the treatment fluid source 514 and to provide the treatment fluid to the intake chamber dosing module 512. The treatment fluid pump 516 is used to pressurize the treatment fluid from the treatment fluid source 514 for delivery to the intake chamber dosing module 512. In some embodiments, the treatment fluid pump 516 is pressure controlled. In some embodiments, the treatment fluid pump 516 is coupled to a chassis of a vehicle associated with the aftertreatment system 500.

In some embodiments, the treatment fluid delivery system 510 also includes a treatment fluid filter 518. The treatment fluid filter 518 is configured to receive the treatment fluid from the treatment fluid source 514 and to provide the treatment fluid to the treatment fluid pump 516. The treatment fluid filter 518 filters the treatment fluid prior to the treatment fluid being provided to internal components of the treatment fluid pump 516. For example, the treatment fluid filter 518 may inhibit or prevent the transmission of solids to the internal components of the treatment fluid pump 516. In this way, the treatment fluid filter 518 may facilitate prolonged desirable operation of the treatment fluid pump 516.

The intake chamber dosing module 512 includes at least one intake chamber dosing module injector 520 (e.g., insertion device, etc.). The intake chamber dosing module injector 520 is configured to receive the treatment fluid from the treatment fluid pump 516. The intake chamber dosing module injector 520 is configured to dose (e.g., provide, inject, insert, etc.) the treatment fluid received by the intake chamber dosing module 512 into the exhaust gas within the intake chamber 508.

In some embodiments, the treatment fluid delivery system 510 also includes an air pump 522 and an air source 524 (e.g., air intake, etc.). The air pump 522 is configured to receive air from the air source 524. The air pump 522 is configured to provide the air to the intake chamber dosing module 512. In some applications, the intake chamber dosing module 512 is configured to mix the air and the treatment fluid into an air-treatment fluid mixture and to provide the air-treatment fluid mixture to the intake chamber dosing module injector 520 (e.g., for dosing into the exhaust gas within the intake chamber 508, etc.). As used herein, it is understood that a treatment fluid may include or consistent of an air-treatment fluid mixture.

The intake chamber dosing module injector 520 is configured to receive the air from the air pump 522. The intake chamber dosing module injector 520 is configured to dose the air into the exhaust gas within the intake chamber 508. In some of these embodiments, the treatment fluid delivery system 510 also includes an air filter 526. The air filter 526 is configured to receive the air from the air source 524 and to provide the air to the air pump 522. The air filter 526 is configured to filter the air prior to the air being provided to the air pump 522. In other embodiments, the treatment fluid delivery system 510 does not include the air pump 522 and/or the treatment fluid delivery system 510 does not include the air source 524. In such embodiments, the intake chamber dosing module 512 is not configured to mix the treatment fluid with the air.

In various embodiments, the intake chamber dosing module 512 is configured to receive air and fluid, and doses the treatment fluid into the intake chamber 508. In various embodiments, the intake chamber dosing module 512 is configured to receive treatment fluid (and does not receive air), and doses the treatment fluid into the intake chamber 508. In various embodiments, the intake chamber dosing module 512 is configured to receive treatment fluid, and doses the treatment fluid into the intake chamber 508. In various embodiments, the intake chamber dosing module 512 is configured to receive air and treatment fluid, and doses the treatment fluid into the intake chamber 508.

The aftertreatment system 500 also includes an aftertreatment system controller 528 (e.g., control circuit, driver, etc.). The intake chamber dosing module 512, the treatment fluid pump 516, and the air pump 522 are also electrically or communicatively coupled to the aftertreatment system controller 528. The aftertreatment system controller 528 is configured to control the intake chamber dosing module 512 to dose the treatment fluid into the intake chamber 508. The aftertreatment system controller 528 may also be configured to control the treatment fluid pump 516 and/or the air pump 522 in order to control the treatment fluid that is dosed into the intake chamber 508.

The aftertreatment system controller 528 includes an aftertreatment system processing circuit 530. The aftertreatment system processing circuit 530 includes an aftertreatment system processor 532 and an aftertreatment system memory 534. The aftertreatment system processor 532 may include a microprocessor, an application-specific integrated circuit (ASIC), a field-programmable gate array (FPGA), etc., or combinations thereof. The aftertreatment system memory 534 may include, but is not limited to, electronic, optical, magnetic, or any other storage or transmission device capable of providing a processor, ASIC, FPGA, etc. with program instructions. The aftertreatment system memory 534 may include a memory chip, Electrically Erasable Programmable Read-Only Memory (EEPROM), Erasable Programmable Read Only Memory (EPROM), flash memory, or any other suitable memory from which the aftertreatment system controller 528 can read instructions. The instructions may include code from any suitable programming language. The aftertreatment system memory 534 may include various modules that include instructions that are configured to be implemented by the aftertreatment system processor 532.

In various embodiments, the aftertreatment system controller 528 is configured to communicate with a central controller 536 (e.g., engine control unit (ECU), engine control module (ECM), etc.) to control the engine control component 502. The engine control component 502 is configured to control the operation of a component of the internal combustion engine (e.g., throttle valve, fuel valve, air valve, ignition timing circuit, etc.) In some embodiments, the central controller 536 and the aftertreatment system controller 528 are integrated into a single controller.

In some embodiments, the central controller 536 is communicable with a display device (e.g., screen, monitor, touch screen, heads up display (HUD), indicator light, etc.). The display device may be configured to change state in response to receiving information from the central controller 536. For example, the display device may be configured to change between a static state and an alarm state based on a communication from the central controller 536. By changing state, the display device may provide an indication to a user of a status of the treatment fluid delivery system 510.

The aftertreatment system 500 includes an upstream catalyst member 538 (e.g., conversion catalyst member, SCR catalyst member, catalyst metals, etc.). The upstream catalyst member 538 is positioned downstream of the intake chamber 508. The upstream catalyst member 538 is configured to cause decomposition of components of the exhaust gas using the treatment fluid (e.g., via catalytic reactions, etc.). The upstream catalyst member 538 includes an upstream catalyst housing 540. The upstream catalyst housing 540 may be coupled to the intake chamber 508. In some embodiments, the upstream catalyst housing 540 is integrally formed with the intake chamber 508. The upstream catalyst member 538 includes an upstream catalyst substrate 542. The upstream catalyst substrate 542 is coupled to the upstream catalyst housing 540. In some embodiments, the upstream catalyst substrate 542 is integrally formed with the upstream catalyst housing 540.

The upstream catalyst member 538 receives the exhaust gas from the intake chamber 508. The exhaust gas flows through the upstream catalyst substrate 542 and reacts with the so as to cause the exhaust gas to undergo the processes of evaporation, thermolysis, and/or hydrolysis to form non-NOₓ emissions within the introduction conduit 509 and/or the upstream catalyst member 538. In this way the upstream catalyst member 538 is configured to assist the reduction of NOₓ emissions by accelerating a NOₓ reduction process between the reductant and the NOₓ of the exhaust gas into diatomic nitrogen, water, and/or carbon dioxide. The upstream catalyst substrate 542 may include vanadia. Vanadia may be used due to the lengthy deactivation time and the ability to react with the exhaust gas at high temperatures. In some embodiments, vanadia is used because of the benefit of emitting lower N₂O emissions into the environment when exhaust gas temperatures are below 420° C. In some embodiments, the aftertreatment system 500 does not include an upstream catalyst member 538.

The aftertreatment system 500 includes an upstream ammonia slip catalyst substrate 544. The upstream ammonia slip catalyst substrate 544. The upstream ammonia slip catalyst substrate 544 is positioned downstream of the upstream catalyst member 538. In some embodiments, the upstream ammonia slip catalyst substrate 544 is a coating applied to a portion of the outlet of the upstream catalyst member 538. The upstream ammonia slip catalyst substrate 544 is configured to receive the exhaust gas from the upstream catalyst member 538 and assist in the reduction of the byproducts (e.g., ammonia, etc.) of the processes of the intake chamber dosing module 512 and the upstream catalyst member 538. Specifically, the intake chamber dosing module 512 may introduce ammonia into the exhaust gas, however a portion of the ammonia introduced may not react with the exhaust gas. As a result, excess ammonia may slip from the upstream catalyst member 538 into the exhaust gas downstream of the upstream catalyst member 538 such that the exhaust gas downstream of the upstream ammonia slip catalyst substrate 544 does not contain an undesirable amount of ammonia. In some embodiments, the aftertreatment system 500 does not include the upstream ammonia slip catalyst substrate 544.

The aftertreatment system 500 includes a first hydrocarbon decomposition chamber 546. The first hydrocarbon decomposition chamber is positioned downstream of the upstream ammonia slip catalyst substrate 544. In some embodiments the first hydrocarbon decomposition chamber 546 is coupled to the upstream catalyst housing 540. In some embodiments, the hydrocarbon decomposition chamber is integrally formed with the upstream catalyst housing 540. In still other embodiments, the first hydrocarbon decomposition chamber 546 is coupled to the intake chamber 508. In some embodiments, the aftertreatment system 500 does not include the upstream catalyst member 538 such that the first hydrocarbon decomposition chamber 546 may also be integrally formed with the intake chamber 508. The first hydrocarbon decomposition chamber 546 is configured to receive the exhaust gas from the upstream ammonia slip catalyst substrate 544. The aftertreatment system 500 includes a hydrocarbon fluid system 547. The hydrocarbon fluid system 547 includes a first hydrocarbon dosing module 548. The first hydrocarbon dosing module 548 doses the exhaust gas within the first hydrocarbon decomposition chamber 546 with hydrocarbons. The first hydrocarbon dosing module 548 is configured to facilitate passage of hydrocarbon through the first hydrocarbon decomposition chamber 546 and into the first hydrocarbon decomposition chamber 546. The first hydrocarbon dosing module 548 includes at least one first hydrocarbon injector 550 (e.g., insertion device, etc.). The first hydrocarbon injector 550 is configured to dose the hydrocarbons into the exhaust gas within the first hydrocarbon decomposition chamber 546.

The hydrocarbons within the first hydrocarbon decomposition chamber 546 may be configured to increase the temperature of the exhaust gas within the first hydrocarbon decomposition chamber 546. Specifically, the aftertreatment system 300 includes a first igniter 551 (e.g., spark plug, etc.) coupled to the first hydrocarbon decomposition chamber 346. The first igniter 551 is electrically connected to the aftertreatment system controller 328 and is configured to combust the hydrocarbons within the first hydrocarbon decomposition chamber 546 which causes the increase in temperature of the exhaust gas. By this way, regeneration of downstream components may occur.

The hydrocarbon fluid system 547 further includes a hydrocarbon source 552 (e.g., hydrocarbon tank, etc.). The hydrocarbon source 552 is configured to contain hydrocarbon. The hydrocarbon source 552 is configured to provide hydrocarbons to the first hydrocarbon dosing module 548. The hydrocarbon source 552 may include multiple hydrocarbon sources 552 (e.g., multiple tanks connected in series or in parallel, etc.). The treatment fluid delivery system also includes a hydrocarbon pump 554. Specifically, the hydrocarbon pump 554 is configured to provide hydrocarbons to the first hydrocarbon injector 550. The first hydrocarbon injector 550 receives the hydrocarbons from the hydrocarbon pump 554 and is configured to dose the hydrocarbon received by the first hydrocarbon dosing module 548 into the exhaust gas within the first hydrocarbon decomposition chamber 546. The hydrocarbon pump 554 is used to pressurize hydrocarbons from the hydrocarbon source 552 for delivery to the first hydrocarbon dosing module 548 and the first hydrocarbon injector 550. In some embodiments, the hydrocarbon pump 554 is pressure controlled. In some embodiments the hydrocarbon pump 554 is coupled to a chassis of a vehicle associated with the aftertreatment system.

In some embodiments, the hydrocarbon fluid system 547 includes a hydrocarbon filter 556 (e.g., fuel filter, lubricant filter, oil filter, etc.). The hydrocarbon filter 556 is configured to receive the hydrocarbons from the hydrocarbon source 552 and to provide the hydrocarbons to the hydrocarbon pump 554. The hydrocarbon filter 556 filters the hydrocarbons prior to the hydrocarbons being provided to internal components of the hydrocarbon pump 554. For example, the hydrocarbon filter 556 may inhibit or prevent the transmission of solids to the internal components of the hydrocarbon pump 554. In this way, the hydrocarbon filter 556 may facilitate prolonged desirable operation of the hydrocarbon pump 554.

In some embodiments, the air pump 522 is also configured to provide the air to the first hydrocarbon dosing module 548. The first hydrocarbon dosing module 548 is configured to provide the air into the first hydrocarbon decomposition chamber 546. In some applications, the first hydrocarbon dosing module 548 is configured to mix the air and the hydrocarbons into an air-hydrocarbon fluid mixture and to provide the air-hydrocarbon fluid mixture to the first hydrocarbon injector 550 (e.g., for dosing into the exhaust gas within the first hydrocarbon decomposition chamber 546, etc.).

In various embodiments, the first hydrocarbon dosing module 548 is configured to receive air and hydrocarbon, and doses the hydrocarbons into the first hydrocarbon decomposition chamber 546. In various embodiments, the first hydrocarbon dosing module 548 is configured to receive hydrocarbons (and does not receive air), and doses the hydrocarbon into the intake chamber 508. In various embodiments, the first hydrocarbon dosing module 548 is configured to receive hydrocarbons, and doses the hydrocarbon into the first hydrocarbon decomposition chamber 546.

In some embodiments, the first hydrocarbon dosing module 548 and the hydrocarbon pump 554 are also electrically or communicatively coupled to the aftertreatment system controller 528. The aftertreatment system controller 528 is further configured to control the first hydrocarbon dosing module 548 to dose the hydrocarbon into the first hydrocarbon decomposition chamber 546. The aftertreatment system controller 528 may also be configured to control the hydrocarbon pump 554 and/or the air pump 522 in order to control the hydrocarbon that is dosed into the first hydrocarbon decomposition chamber 546. In some embodiments, the aftertreatment system 500 does not include the first hydrocarbon decomposition chamber 546, the first hydrocarbon dosing module 548, the first hydrocarbon injector 550, the hydrocarbon source 552, the hydrocarbon pump 554, and/or the hydrocarbon filter 556.

The aftertreatment system 500 includes a first oxidation catalyst member 558 (e.g., first DOC). The first oxidation catalyst member 558 is positioned downstream of first hydrocarbon decomposition chamber 546 (i.e., the first hydrocarbon decomposition chamber 546 is positioned upstream of the first oxidation catalyst member 558). The first oxidation catalyst member includes a first oxidation catalyst housing 560. The first oxidation catalyst housing 560 is coupled to first hydrocarbon decomposition chamber 546. The first oxidation catalyst housing 560 may also be integrally formed with first hydrocarbon decomposition chamber 546.

The first oxidation catalyst member 558 includes a first oxidation catalyst substrate 562 (e.g., DOC, etc.). The first oxidation catalyst substrate 562 is positioned within the first oxidation catalyst housing 560. The first oxidation catalyst substrate 562 may be coupled to the first oxidation catalyst housing 560. The exhaust gas including the hydrocarbons react with the first oxidation catalyst substrate 562 and cause the conversion of the hydrocarbons. For example, as the exhaust gas flows the through the first oxidation catalyst substrate 562, the hydrocarbons react with the first oxidation catalyst substrate 562. The first oxidation catalyst substrate 562 facilitates conversion of the carbon monoxide in the exhaust gas and the hydrocarbons and/or the air-hydrocarbon mixture into carbon dioxide.

In some embodiments, the aftertreatment system 500 does not include the upstream catalyst member 538, the upstream ammonia slip catalyst substrate 544, and/or the first hydrocarbon decomposition chamber 546 such that the first oxidation catalyst member 558 may be positioned downstream of the intake chamber 508. In such embodiments, the first oxidation catalyst housing 560 may be coupled to the intake chamber 508. In other such embodiments, the first oxidation catalyst housing 560 may also be integrally formed with the intake chamber 508.

The aftertreatment system 500 also includes an upstream particulate filter assembly 564. The upstream particulate filter assembly 564 includes an upstream particulate filter housing 566 and an upstream particulate filter 568 (e.g., DPF, filtration member, etc.). The upstream particulate filter housing 566 is positioned downstream of the first oxidation catalyst housing 560. In some embodiments, the upstream particulate filter housing 566 is integrally formed with the first oxidation catalyst housing 560. The upstream particulate filter 568 is disposed within the upstream particulate filter housing 566 such that the upstream particulate filter 568 is positioned downstream of the first oxidation catalyst member 558 (i.e., the first oxidation catalyst member 558 is positioned upstream of the upstream particulate filter 568). In some embodiments, the upstream particulate filter housing 566 and the upstream particulate filter 568 are positioned downstream of the intake chamber 508.

The upstream particulate filter 568 is configured to remove first particulates (e.g., soot, solidified hydrocarbons, ash, etc.) from the exhaust gas. For example, the upstream particulate filter 568 may receive exhaust gas (e.g., from the first oxidation catalyst member 558, the intake chamber 508 etc.) having a first concentration of the first particulates and may provide the exhaust gas downstream having a second concentration of the first particulates, where the second concentration is lower than the first concentration. In some embodiments, the upstream particulate filter 568 is a catalyzed DPF. In this way, the upstream particulate filter 568 may facilitate reduction of a particulate number (PN) of the exhaust gas. Decreasing the PN of the exhaust gas may be desirable in a variety of applications. For example, emissions regulations may prescribe a maximum PN for exhaust gas emitted to atmosphere and the upstream particulate filter 568 may ensure that the PN of the exhaust gas emitted to atmosphere by the aftertreatment system 500 is below the maximum PN.

In some embodiments, the upstream particulate filter 568 is a catalyzed DPF. The catalyzed DPF is a filter that has a catalyst coating. The catalyst coating is configured to react with a component of the exhaust gas to reduce undesirable components in the exhaust. For example, the catalyst coating could be an oxidation catalyst to reduce hydrocarbons within the exhaust gas. In some embodiments, the catalyst coating is a SCR catalyst configured to reduce NOₓ emissions.

The aftertreatment system 500 includes a decomposition chamber 570 (e.g., decomposition reactor, decomposition reactor tube (DRT), etc.). The decomposition chamber 570 is positioned downstream of the upstream particulate filter assembly 564 (i.e., the decomposition chamber 570 is positioned downstream of the upstream particulate filter 568) and configured to receive exhaust gas from the upstream particulate filter assembly 564. The decomposition chamber 570 may be coupled to the upstream particulate filter housing 566. In some embodiments, the decomposition chamber 570 is integrally formed with the upstream particulate filter housing 566.

The treatment fluid delivery system 510 includes a decomposition chamber dosing module 572. The decomposition chamber dosing module 572 is configured to facilitate passage of the treatment fluid through the decomposition chamber 570 and into the decomposition chamber 570. In some embodiments, the decomposition chamber dosing module 572 is positioned within a dosing module mount. The dosing module mount is configure to facilitate mounting of the decomposition chamber dosing module 572 to the decomposition chamber 570. The dosing module mount may provide insulation (e.g., thermal insulation, vibrational insulation, etc.) between the decomposition chamber dosing module 572 and the decomposition chamber.

The decomposition chamber dosing module 572 includes at least one decomposition chamber injector 574 (e.g., insertion device, etc.). The decomposition chamber injector 574 is configured to receive the treatment fluid from the treatment fluid pump 516. The decomposition chamber injector 574 is configured to dose the treatment fluid received by the decomposition chamber dosing module 572 into the exhaust gas within the decomposition chamber 570. When the treatment fluid is introduced into the exhaust gas, reduction of emission of undesirable particulates in the exhaust gas may be facilitated.

In some embodiments, the decomposition chamber dosing module 572 is configured to receive air from the air pump 522. In some applications, the decomposition chamber dosing module 572 is configured to mix the air and the treatment fluid into an air-treatment fluid mixture and to provide the air-treatment fluid mixture to the decomposition chamber injector 574 (e.g., for dosing into the exhaust gas within the decomposition chamber 570, etc.). Specifically, the decomposition chamber injector 574 is configured to receive the air from the air pump 522. The intake chamber dosing module injector 520 is configured to dose the air into the exhaust gas within the decomposition chamber 570.

In various embodiments, the decomposition chamber dosing module 572 is configured to receive air and fluid, and doses the treatment fluid into the decomposition chamber 570. In various embodiments, the decomposition chamber dosing module 572 is configured to receive treatment fluid (and does not receive air), and doses the treatment fluid into the decomposition chamber 570. In various embodiments, the decomposition chamber dosing module 572 is configured to receive treatment fluid, and doses the treatment fluid into the decomposition chamber 570. In various embodiments, the decomposition chamber dosing module 572 is configured to receive air and treatment fluid, and doses the treatment fluid into the decomposition chamber 570.

The aftertreatment system 500 includes a first downstream catalyst member 576 (e.g., conversion catalyst member, SCR catalyst member, catalyst metals, etc.). The first downstream catalyst member 576 is positioned downstream of the decomposition chamber 570. The first downstream catalyst member 576 is configured to cause decomposition of components of the exhaust gas using the treatment fluid (e.g., via catalytic reactions, etc.). The first downstream catalyst member 576 includes a first downstream catalyst housing 578 and a first downstream catalyst substrate 580. The first downstream catalyst housing 578 may be coupled to the decomposition chamber 570. In some embodiments, the first downstream catalyst housing 578 is integrally formed with the decomposition chamber 570. The first downstream catalyst substrate 580 is coupled to the first downstream catalyst housing 578. In some embodiments, the first downstream catalyst substrate 580 is integrally formed with the first downstream catalyst housing 578.

The first downstream catalyst member 576 receives the exhaust gas from the decomposition chamber 570. The exhaust gas flows through the first downstream catalyst substrate 580 and reacts with the first downstream catalyst substrate 580 so as to cause the exhaust gas to undergo the processes of evaporation, thermolysis, and/or hydrolysis to form non-NOₓ emissions within the introduction conduit 509 and/or the first downstream catalyst member 576. In some embodiments, the exhaust gas and the treatment fluid within the exhaust gas react with the first downstream catalyst substrate 580. In this way the first downstream catalyst member 576 is configured to assist the reduction of NOₓ emissions by accelerating a NOₓ reduction process between the reductant and the NOₓ of the exhaust gas into diatomic nitrogen, water, and/or carbon dioxide and also configured to assist in the reduction of particulates from the exhaust gas. The first downstream catalyst member 576 may include iron zeolite. The first downstream catalyst member 576 may include copper zeolite. In some embodiments, the aftertreatment system 500 does not include a first downstream catalyst member 576.

The aftertreatment system 500 includes a second downstream catalyst member 582 (e.g., conversion catalyst member, SCR catalyst member, catalyst metals, etc.). The second downstream catalyst member 582 is positioned downstream of the decomposition chamber 570. The second downstream catalyst member 582 is configured to cause decomposition of components of the exhaust gas using the treatment fluid (e.g., via catalytic reactions, etc.). The second downstream catalyst member 582 includes a second downstream catalyst housing 584. In some embodiments, the second downstream catalyst housing 584 is integrally formed with the first downstream catalyst housing 578. In some embodiments, the second downstream catalyst housing 584 is the first downstream catalyst housing 578. The second downstream catalyst member 582 also includes a second downstream catalyst substrate 586. The second downstream catalyst substrate 586 is coupled to the second downstream catalyst housing 584. In some embodiments, the second downstream catalyst substrate 586 is integrally formed with the second downstream catalyst housing 584.

The second downstream catalyst member 582 receives the exhaust gas from the first downstream catalyst member 576. The exhaust gas flows through the second downstream catalyst substrate 586 and reacts with the second downstream catalyst substrate 586 so as to cause the exhaust gas to undergo the processes of evaporation, thermolysis, and/or hydrolysis to form non-NOₓ emissions within the introduction conduit 509 and/or the second downstream catalyst member 582. In some embodiments, the exhaust gas and the treatment fluid within the exhaust gas react with the second downstream catalyst substrate 586. In this way the second downstream catalyst member 582 is configured to assist the reduction of NOₓ emissions by accelerating a NOₓ reduction process between the reductant and the NOₓ of the exhaust gas into diatomic nitrogen, water, and/or carbon dioxide and also configured to assist in the reduction of particulates from the exhaust gas. The second downstream catalyst member 582 may include iron zeolite. The second downstream catalyst member 582 may include copper zeolite. In some embodiments, the aftertreatment system 500 does not include a second downstream catalyst member 582.

The aftertreatment system 500 also includes a downstream particulate filter assembly 588. The downstream particulate filter assembly 588 includes a downstream particulate filter housing 590 and a downstream particulate filter 592 (e.g., DPF, filtration member, catalyzed filter member, selective catalyst reduction filter, etc.). The downstream particulate filter housing 590 is positioned downstream of the second downstream catalyst member 582. In some embodiments, the downstream particulate filter housing 590 is integrally formed with the second downstream catalyst housing 584. The downstream particulate filter 592 is disposed within the downstream particulate filter housing 590 such that the downstream particulate filter 592 is positioned downstream of the second downstream catalyst member 582. In some embodiments, the downstream particulate filter housing 590 is positioned downstream of the first downstream catalyst member 576. In some embodiments, the downstream particulate filter housing 590 is integrally formed with the first downstream catalyst housing 578. The downstream particulate filter 592 is disposed within the downstream particulate filter housing 590 such that the downstream particulate filter 592 is positioned downstream of the first downstream catalyst member 576.

The downstream particulate filter 592 includes a filter substrate 594. The downstream particulate substrate is configured to remove second particulates from the exhaust gas. For example, the downstream particulate filter 592 may receive exhaust gas (e.g., from the first downstream catalyst member 576, the second downstream catalyst member 582, etc.) having a first concentration of the second particulates, the filter substrate 594 facilitates inhibition of a portion of the second particulates and facilitates the exhaust gas downstream having a second concentration of the second particulates, where the second concentration is lower than the first concentration. In this way, the downstream particulate filter 194 may facilitate reduction of the particulate number within the exhaust gas Decreasing the PN of the exhaust gas may be desirable in a variety of applications. For example, emissions regulations may prescribe a maximum PN for exhaust gas emitted to atmosphere and the upstream particulate filter 168 may ensure that the PN of the exhaust gas emitted to atmosphere by the aftertreatment system 100 is below the maximum PN. The filter substrate 594 of the downstream particulate filter 380 includes a catalyst coating. The catalyst coating may be applied to a portion of the inlet and the outlet of the filter substrate. In some embodiments the catalyst coating comprises copper zeolite.

The aftertreatment system 500 includes a third downstream catalyst member 596 (e.g., conversion catalyst member, SCR catalyst member, catalyst metals, etc.). The third downstream catalyst member 596 is positioned downstream of the downstream particulate filter assembly 588 (e.g., the third downstream catalyst member 596 is positioned downstream of downstream particulate filter 592). The third downstream catalyst member 596 is configured to cause decomposition of components of the exhaust gas using the treatment fluid (e.g., via catalytic reactions, etc.). The third downstream catalyst member 596 includes a third downstream catalyst housing 598 and a third downstream catalyst substrate 600. The third downstream catalyst housing 598 may be coupled to the downstream particulate filter housing 590. In some embodiments, the third downstream catalyst housing 598 is integrally formed with the downstream particulate filter housing 590. The third downstream catalyst substrate 600 is coupled to the third downstream catalyst housing 598. In some embodiments, the third downstream catalyst substrate 600 is integrally formed with the third downstream catalyst housing 598.

The third downstream catalyst member 596 receives the exhaust gas from the downstream particulate filter assembly 588. The exhaust gas flows through the third downstream catalyst substrate 600 and reacts with the third downstream catalyst substrate 600 so as to cause the exhaust gas to undergo the processes of evaporation, thermolysis, and/or hydrolysis to form non-NOₓ emissions within the introduction conduit 509 and/or the third downstream catalyst member 596. In some embodiments, the exhaust gas and the treatment fluid in the exhaust gas react with the third downstream catalyst substrate 600. In this way the third downstream catalyst member 596 is configured to assist the reduction of NOₓ emissions by accelerating a NOₓ reduction process between the reductant and the NOₓ of the exhaust gas into diatomic nitrogen, water, and/or carbon dioxide and also configured to assist in the reduction of particulates from the exhaust gas. The first downstream catalyst member 386 may include iron zeolite. The first downstream catalyst member 386 may include copper zeolite. In some embodiments, the aftertreatment system 500 does not include a third downstream catalyst member 596.

The aftertreatment system 500 includes a downstream ammonia slip catalyst substrate 602. The downstream ammonia slip catalyst substrate 602 is positioned downstream of the third downstream catalyst member 596. In some embodiments, the downstream ammonia slip catalyst substrate 602 is a coating applied to the outlet of the third downstream catalyst member 596. In other embodiments, the downstream ammonia slip catalyst substrate 602 is a coating applied to a portion of the outlet of the first downstream catalyst member 576. The downstream ammonia slip catalyst substrate 602 may be a coating applied to a portion of the outlet of the second downstream catalyst member 582. The downstream ammonia slip catalyst substrate 602 is configured to receive the exhaust gas from the third downstream catalyst member 596 and assist in the reduction of the byproducts (e.g., ammonia, etc.) of the processes of the decomposition chamber dosing module 572, the first downstream catalyst member 576, the second downstream catalyst member 582, and/or the third downstream catalyst member 596. Specifically, the decomposition chamber dosing module 572 may introduce ammonia into the exhaust gas, however a portion of the ammonia introduced may not react with the exhaust gas. As a result, the excess ammonia may slip from the first downstream catalyst member 576, the second downstream catalyst member 582, and/or the third downstream catalyst member 596 into the exhaust gas downstream of the first downstream catalyst member 576, the second downstream catalyst member 582, and/or the third downstream catalyst member 596 such that the exhaust gas downstream of the downstream ammonia slip catalyst substrate 602 does not contain an undesirable amount of ammonia. In some embodiments, the aftertreatment system 100 does not include the downstream ammonia slip catalyst substrate 602.

The aftertreatment system 500 also includes an outlet chamber 604. The outlet chamber 604 is positioned downstream of the downstream particulate filter assembly 588 and is configured to receive the exhaust gas from the downstream particulate filter 592. In various embodiments, the outlet chamber 604 is coupled to the downstream particulate filter housing 590. For example, the outlet chamber 604 may be fastened, welded, riveted, or otherwise attached to the downstream particulate filter housing 590. In other embodiments, the outlet chamber 604 is integrally formed with the downstream particulate filter housing 590. In some embodiments, the outlet chamber 604 is coupled to the introduction conduit 509. In some embodiments, the outlet chamber 604 is the introduction conduit 509 (e.g., only the introduction conduit is included in the exhaust gas conduit system 504 and the introduction conduit 509 functions as both the introduction conduit 509 and the outlet chamber 604). The outlet chamber 604 is centered on the conduit center axis 506 (e.g., the conduit center axis 506 extends through a center point of the outlet chamber 604, etc.).

In various embodiments, the exhaust gas conduit system 504 only includes a single conduit that functions as the intake chamber 508, the introduction conduit 509, and the outlet chamber 604.

In various embodiments, the aftertreatment system 500 also includes a first sensor 606 (e.g., NOₓ sensor, CO sensor, CO₂ sensor, O₂ sensor, particulate sensor, nitrogen sensor, etc.). The first sensor 606 is positioned downstream of the downstream particulate filter 592. In some embodiments, the first sensor 606 is coupled to the outlet chamber 604. The first sensor 606 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid downstream of the downstream particulate filter 592. The first sensor 606 may be configured to measure the parameter within the outlet chamber 604. In some embodiments, the parameter measured by the first sensor 606 is the particulate concentration in the exhaust gas downstream of the downstream particulate filter 592. In some embodiments, the parameter measured by the first sensor 606 is the SOₓ concentration of the exhaust gas within the outlet chamber 604. In some embodiments, the first sensor 606 measures both the particulate concentration and the SOₓ concentration.

The first sensor 606 is electrically or communicatively coupled to the aftertreatment system controller 528 and is configured to provide a first signal associated with the parameter to the aftertreatment system controller 528. The aftertreatment system controller 528 (e.g., via the aftertreatment system processing circuit 530, etc.) is configured to determine a first measurement based on the first signal. The aftertreatment system controller 528 may be configured to control the intake chamber dosing module 512, the decomposition chamber dosing module 572, the treatment fluid pump 516, and/or the air pump 522 based on the first signal. Furthermore, the aftertreatment system controller 528 may be configured to communicate the first signal to the central controller 536.

In various embodiments, the aftertreatment system 500 also includes a second sensor 608 (e.g., NOₓ sensor, CO sensor, CO₂ sensor, O₂ sensor, particulate sensor, nitrogen sensor, etc.). The second sensor 608 is positioned downstream of the downstream particulate filter 592. In some embodiments, the second sensor 608 is coupled to the outlet chamber 604 and positioned downstream of the first sensor 606. The second sensor 608 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid downstream of the downstream particulate filter 592. The second sensor 608 may be configure to measure the parameter of the exhaust gas within the outlet chamber 604. In some embodiments, the parameter measured by the second sensor 608 is the particulate concentration in the exhaust gas downstream of the downstream particulate filter 592. In some embodiments, the parameter measured by the second sensor 608 is the SOₓ concentration of the exhaust gas downstream of the downstream particulate filter 592. In some embodiments, the second sensor 608 measures both the particulate concentration and the SOₓ concentration.

The second sensor 608 is electrically or communicatively coupled to the aftertreatment system controller 528 and is configured to provide a second signal associated with the parameter to the aftertreatment system controller 528. The aftertreatment system controller 528 (e.g., via the aftertreatment system processing circuit 530, etc.) is configured to determine a second measurement based on the second signal. The aftertreatment system controller 528 may be configured to control the intake chamber dosing module 512, the decomposition chamber dosing module 572, the treatment fluid pump 516, and/or the air pump 522 based on the second signal. Furthermore, the aftertreatment system controller 528 may be configured to communicate the second signal to the central controller 536.

In various embodiments, the aftertreatment system 500 also includes a third sensor 610 (e.g., NOₓ sensor, CO sensor, CO₂ sensor, O₂ sensor, particulate sensor, nitrogen sensor, etc.). The third sensor 610 is positioned upstream of the upstream particulate filter 568. In some embodiments, the third sensor 610 is coupled to the intake chamber 508. The third sensor 610 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid upstream of the upstream particulate filter 568. The third sensor 610 may be configured to measure a parameter of the exhaust gas within the intake chamber 508. In some embodiments, the parameter measured by the first sensor 606 is the particulate concentration in the exhaust gas upstream of the upstream particulate filter 568. In some embodiments, the parameter measured by the third sensor 610 is the SOₓ concentration of the exhaust gas upstream of the upstream particulate filter 568. In some embodiments, the third sensor 610 measures both the particulate concentration and the SOₓ concentration.

The third sensor 610 is electrically or communicatively coupled to the aftertreatment system controller 528 and is configured to provide a third signal associated with the parameter to the aftertreatment system controller 528. The aftertreatment system controller 528 (e.g., via the aftertreatment system processing circuit 530, etc.) is configured to determine a third measurement based on the third signal. The aftertreatment system controller 528 may be configured to control the intake chamber dosing module 512, the decomposition chamber dosing module 572, the treatment fluid pump 516, and/or the air pump 522 based on the third signal. Furthermore, the aftertreatment system controller 528 may be configured to communicate the third signal to the central controller 536.

In various embodiments, the aftertreatment system 500 also includes a fourth sensor 612 (e.g., NOₓ sensor, CO sensor, CO₂ sensor, O₂ sensor, particulate sensor, nitrogen sensor, etc.). The fourth sensor 612 is positioned upstream of the upstream particulate filter 568. In some embodiments, the fourth sensor 612 is coupled to the intake chamber 508 and positioned downstream of the third sensor 610. The fourth sensor 612 is configured to measure (e.g., sense, detect, etc.) a parameter (e.g., NOₓ concentration, CO concentration, CO₂ concentration, O₂ concentration, particulate concentration, nitrogen concentration, SOₓ etc.) of the exhaust gas and the treatment fluid upstream of the upstream particulate filter 568. The fourth sensor 612 may be configured to measure a parameter of the exhaust gas upstream of the upstream particulate filter 568. In some embodiments, the parameter measured by the fourth sensor 612 is the particulate concentration in the exhaust gas upstream of the upstream particulate filter 568. In some embodiments, the parameter measured by the fourth sensor 612 is the SOₓ concentration of the exhaust gas upstream of the upstream particulate filter 568. In some embodiments, the fourth sensor 612 measures both the particulate concentration and the SOₓ concentration.

The fourth sensor 612 is electrically or communicatively coupled to the aftertreatment system controller 528 and is configured to provide a fourth signal associated with the parameter to the aftertreatment system controller 528. The aftertreatment system controller 528 (e.g., via the aftertreatment system processing circuit 530, etc.) is configured to determine a fourth measurement based on the fourth signal. The aftertreatment system controller 528 may be configured to control the intake chamber dosing module 512, the decomposition chamber dosing module 572, the treatment fluid pump 516, and/or the air pump 522 based on the fourth signal. Furthermore, the aftertreatment system controller 528 may be configured to communicate the fourth signal to the central controller 536.

In various embodiments, the aftertreatment system 500 includes a fifth sensor 614 (e.g., NOₓ sensor, CO sensor, CO₂ sensor, O₂ sensor, particulate sensor, nitrogen sensor, etc.). The fifth sensor 614 is configured substantially similar to the fifth sensor 206 and therefore not described in further detail.

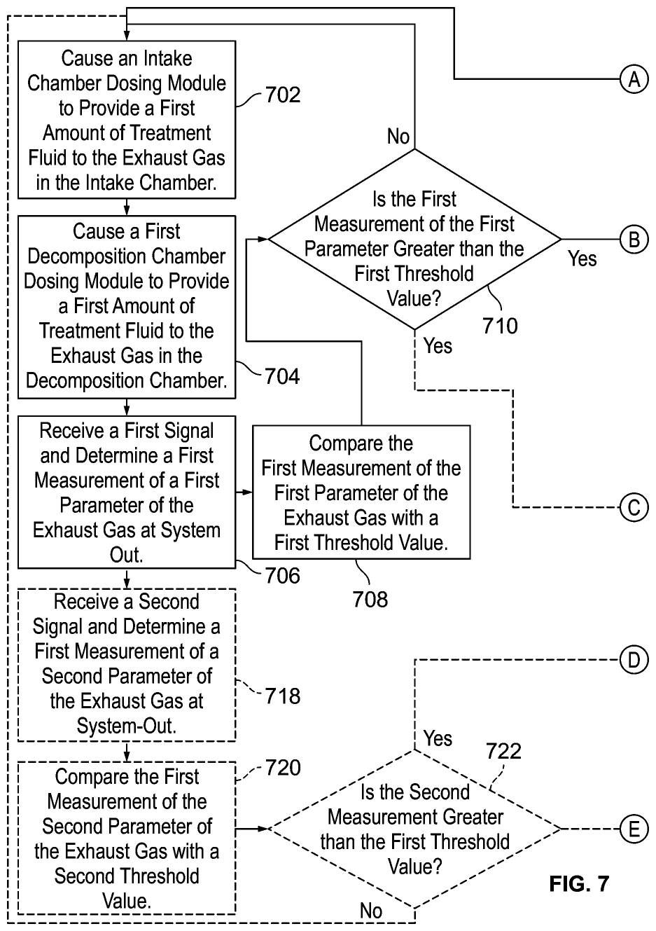

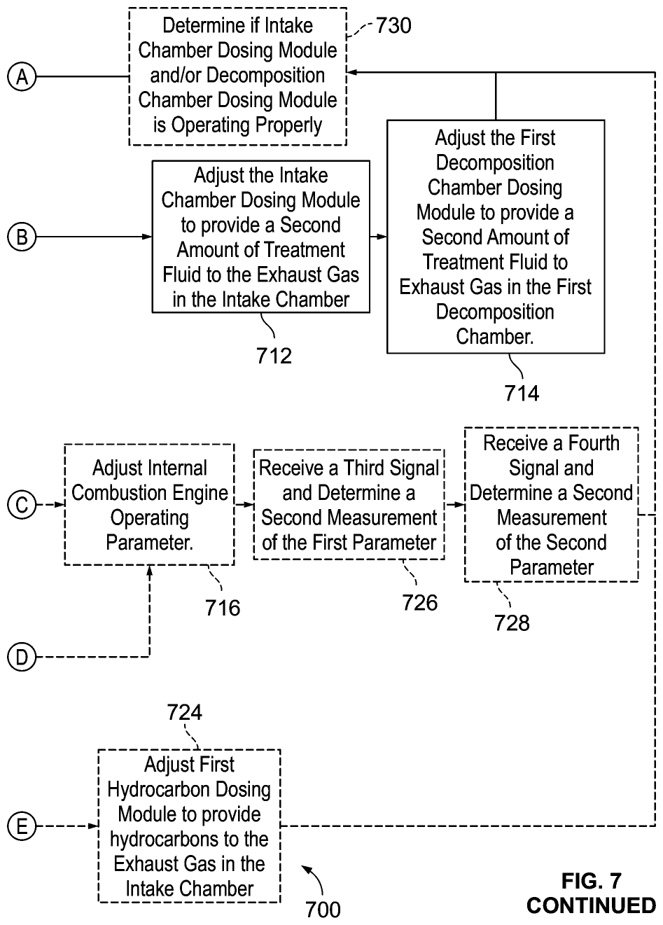

FIG. 7 illustrates an aftertreatment system control strategy 700 (e.g., method, process, etc.). The aftertreatment system control strategy 700 is implemented by an aftertreatment system controller (e.g., the aftertreatment system controller 128, the aftertreatment system controller 328, the aftertreatment system controller 528, etc.) of an aftertreatment system (e.g., the aftertreatment system 100, the aftertreatment system 300, the aftertreatment system 500, etc.). Specifically, the aftertreatment system control strategy 700 is implemented by the aftertreatment system controller and utilizes inputs from various sensors (e.g., the first sensor 198, the second sensor 200, the third sensor 202, the fourth sensor 204, fifth sensor 206, the first sensor 398, the second sensor 400, the third sensor 402, the fourth sensor 404, the fifth sensor 406, the first sensor 606, the second sensor 608, the third sensor 610, the fourth sensor 612, the fifth sensor 614, etc.) to adjust operation of the aftertreatment system 100, 300, 500 etc. and/or an internal combustion engine associated with the aftertreatment system 100, 300, 500, etc., so as to control emission of undesirable components in the exhaust gas.

In normal operation of the aftertreatment system 100, where the intake chamber dosing module 112 and the decomposition chamber dosing module 172 are dosing the correct amount of treatment fluid to the exhaust gases (e.g. as opposed to dosing too much treatment fluid or too little treatment fluid, etc.), a system-out NOₓ (SONOₓ) measurement obtained from the first sensor 198 and an engine-out NOₓ (EONOₓ) obtained by the third sensor 202 generally change together (e.g. increases in EONOₓ measurements generally correspond to increases in SONOₓ measurements and decreases in EONOₓ measurements generally correspond to decreases in SONOₓ measurements). The aftertreatment system control strategy 700 operates to detect instances where the SONOₓ measurement changes more or less than expected, based on changes in EONOₓ measurements, in order to determine if the intake chamber dosing module (e.g., intake chamber dosing module 112, intake chamber dosing module 312, intake chamber dosing module 512, etc.) and/or the decomposition chamber dosing module (e.g., decomposition chamber dosing module 172, decomposition chamber dosing module 372, decomposition chamber dosing module 572, etc.) are dosing too much or too little treatment fluid. If the aftertreatment system controller determines that the intake chamber dosing module and/or the decomposition chamber dosing module are doing too much or too little treatment fluid, the aftertreatment system control strategy 700 then operates to correct the amount of the treatment fluid the intake chamber dosing module and/or the decomposition chamber dosing module is dosing to prevent any over-dosing or under-dosing. In various embodiments, the aftertreatment system control strategy 700 also operates to detect instances where the SONOₓ measurement changes more or less than expected, based on changes in EONOₓ measurements and the treatment fluid flow rates of the intake chamber dosing module and/or the decomposition chamber dosing module, in order to determine if the intake chamber dosing module and/or the decomposition chamber dosing module are providing the commanded dosing rate.

The aftertreatment system control strategy begins in block 702 with causing, by the aftertreatment system controller, the intake chamber dosing module to provide a first amount of treatment fluid to the exhaust gas into an intake chamber. The aftertreatment system control strategy continues to block 704 with causing, by the aftertreatment system controller the decomposition chamber dosing module to provide a first amount of treatment fluid to the exhaust gas into the decomposition chamber. In some embodiments, the first amount of treatment fluid provided to the exhaust gas in the intake chamber is approximately equal to (e.g., within 5% of being equal to, etc.) the first amount of treatment fluid provided to the exhaust gas in the decomposition chamber. In other embodiments, the first amount of treatment fluid provided to the exhaust gas in the decomposition chamber is greater than or less than the first amount of treatment fluid provided to the exhaust gas in the intake chamber (e.g. the first amount of treatment fluid provided to the exhaust gas in the decomposition chamber is greater than the first amount of treatment fluid provided to the exhaust gas in the intake chamber, the first amount of treatment fluid provided to the exhaust gas in the decomposition chamber is less than the first amount of treatment fluid provided to the exhaust gas in the intake chamber, etc.)

The aftertreatment system control strategy continues in block 706, with determining, by the aftertreatment system controller a first measurement of a first parameter of the exhaust gas at system-out. Specifically, the first sensor (e.g., first sensor 198, first sensor 398, first sensor 606, etc.) obtains a first signal corresponding to the first parameter of the exhaust gas in the outlet chamber (e.g., outlet chamber 196, outlet chamber 396, outlet chamber 604, etc.) and provides the first signal to the aftertreatment system controller. The aftertreatment system controller determines a first measurement of the first parameter (e.g., NOₓSOₓ, particulate concentration, etc.) from the provided first signal at system out. The aftertreatment system controller may store, using the aftertreatment system processor (aftertreatment system processor 132, aftertreatment system processor 332, aftertreatment system processor 532, etc.) the first measurement of the first parameter in the aftertreatment system memory (aftertreatment system memory 134, aftertreatment system memory 334, aftertreatment system memory 534, etc.).

The aftertreatment system control strategy 700 continues in block 708 with comparing, by the aftertreatment system controller, the first measurement of the first parameter with a first threshold value. Specifically, the aftertreatment system controller retrieves the first threshold value (e.g., particulate number threshold, a NOₓ concentration threshold, a SOₓ concentration threshold, etc.) at system-out corresponding to the first parameter from aftertreatment system memory by the aftertreatment system processor. The aftertreatment system controller, by the aftertreatment system processor, compares the first measurement of the first parameter to the first threshold value.

The aftertreatment system control strategy 700 continues to block 710 with determining, by the aftertreatment system controller if the first measurement of the first parameter is greater than the first threshold value.

If the aftertreatment system controller determines that that the first measurement of the first parameter is greater than the first threshold value, the aftertreatment system control strategy continues to block 712 with adjusting, by the aftertreatment system controller, the intake chamber dosing module to provide a second amount of treatment fluid (e.g., different from the first amount of treatment fluid, etc.) to the exhaust gas in the intake chamber 108. The second amount of treatment fluid provided to the exhaust gas in the intake chamber may be greater than the first amount of treatment fluid provided to the exhaust gas in the intake chamber.

In some embodiments, the second amount of treatment fluid provided to the exhaust gas in the intake chamber is a maximum amount able to be provided by the intake chamber dosing module. For example, the aftertreatment system controller operates a valve on the intake chamber dosing module to an open position such that the flow rate of the treatment fluid facilitated from the intake chamber dosing module into the exhaust gas is maximized. By this way, more treatment fluid flows through the aftertreatment system and reacts thereby reducing emissions.

The aftertreatment system control strategy 700 then continues to block 714 with adjusting, by the aftertreatment system controller, the decomposition chamber dosing module to provide a second amount of treatment fluid to the exhaust gas in the decomposition chamber. The second amount of treatment fluid to the exhaust gas in the decomposition chamber may be less than the first amount of treatment fluid provided to the exhaust gas in the decomposition chamber. The second amount of treatment fluid to the exhaust gas in the decomposition chamber may also be less than the second amount of treatment fluid provided to the exhaust gas in the intake chamber. The aftertreatment system control strategy 700 returns to block 702 (e.g., the aftertreatment system control strategy 700 restarts). As the aftertreatment system control strategy 700 returns to block 702, the second amount of treatment fluid to the exhaust gas in the intake chamber becomes first amount of treatment fluid to the exhaust gas in the intake chamber and the second amount of treatment fluid to the exhaust gas in the decomposition chamber is the first amount of treatment fluid to the exhaust gas in the decomposition chamber.

If the aftertreatment system controller determines that the first measurement of the first parameter is not greater than the first threshold value, the aftertreatment system control strategy 700 returns block 702 (e.g., the aftertreatment system control strategy 700 restarts).

In some embodiments, if the aftertreatment system controller determines that the first measurement of the first parameter is greater than the first threshold value at block 710, the aftertreatment system control strategy 700 continues to block 716 with adjusting, by the aftertreatment system controller, one or more engine operating parameters of an internal combustion engine associated with the aftertreatment system.

Specifically at block 716, the aftertreatment system controller communicates with the central controller which communicates with the engine control component 102 to adjust one or more engine operating parameters of the internal combustion engine. For example, the aftertreatment system controller 128 may communicate to the central controller 136 to communicate to the engine control component to adjust the air/fuel ratio by operation of a component of the internal combustion engine. By this way, the aftertreatment system controller 128 may be configured to reduce the EONOₓ and/or the engine-out sulfur dioxide (EOSOₓ). The aftertreatment system control strategy 700 may continue to block 702 (e.g., the aftertreatment system control strategy 700 restarting).

In some embodiments, the aftertreatment system control strategy may continue to block 718, with determining, by the aftertreatment system controller a first measurement of a second parameter of the exhaust gas at system-out. The second parameter is different than the first parameter. Specifically, a second sensor (second sensor 200, second sensor 400, second sensor 608, etc.) obtains a first signal corresponding to the second parameter of the exhaust gas in the outlet chamber and provides the first signal to the aftertreatment system controller. The aftertreatment system controller determines a first measurement of the second parameter (e.g. NOₓ, SOₓ, particulate concentration, etc.) at system-out from the provided second signal. The aftertreatment system controller may store the first measurement of the second parameter onto the aftertreatment system memory. In some embodiments, the first measurement of the second parameter corresponds to the SOₓ in the exhaust gas in the outlet chamber. In some embodiments the second sensor is the same as the first sensor.

The aftertreatment system control strategy 700 may continue to block 720 with comparing, by the aftertreatment system controller, the first measurement of the second parameter of the exhaust gas with a second threshold value. Specifically, the aftertreatment system controller retrieves the second threshold value (e.g., particulate number threshold, a NOₓ concentration threshold, a SOₓ concentration threshold, etc.) at system-out corresponding to a second parameter from aftertreatment system memory by the aftertreatment system processor. The aftertreatment system controller, by the aftertreatment system processor, compares the first measurement of the second parameter to the second threshold value.

The aftertreatment system control strategy 700 continues to block 722 with determining, by the aftertreatment system controller, if the first measurement of the second parameter is greater than the second threshold value.

If the aftertreatment system control strategy 700 determines that the first measurement of the second parameter is greater than the second threshold value, the aftertreatment system control strategy continues to block 716 with adjusting one or more of the internal combustion engine operating parameters. Specifically, the aftertreatment system controller 128 communicates with the central controller 136 to communicate with the engine control component to control operation of a component of the internal combustion engine. Specifically, the central controller 136 adjust the air/fuel ratio of the internal combustion engine. By this way, the temperature of the exhaust gas flowing from the internal combustion engine to the intake chamber is raised which facilitates a regeneration process for the upstream particulate filter (e.g., upstream particulate filter 168, upstream particulate filter 368, upstream particulate filter 568, etc.) and the downstream particulate filter (e.g., downstream particulate filter 380, downstream particulate filter 380, downstream particulate filter 592, etc.).

In some embodiments, if the aftertreatment system control strategy 700 determines that the first measurement of the second parameter is greater than the second threshold value, the aftertreatment system control strategy continues to block 724 with adjusting, by the aftertreatment system controller, the first hydrocarbon dosing module to provide hydrocarbons the exhaust gas in the first hydrocarbon decomposition chamber. Specifically, the aftertreatment system controller increases the amount of hydrocarbons provided such that they may combust and increase the temperature of the exhaust gas within the first hydrocarbon decomposition chamber. By this way, the upstream particulate filter and the downstream particulate filter undergo a regeneration process.

In some embodiments, the aftertreatment system control strategy 700 may continue to block 726 with determining, by the aftertreatment system controller, a second measurement of the first parameter of the exhaust gas at engine-out. Specifically the third sensor (e.g., third sensor 202, third sensor 402, third sensor 610, etc.) obtains a third signal corresponding to the first parameter of the exhaust gas in the intake chamber and provides the third signal to the aftertreatment system controller. The aftertreatment system controller determines a second measurement of the first parameter (e.g. NOₓ, SOₓ, particulate concentration, etc.) from the provided third signal at the engine-out. The aftertreatment system controller may store the second measurement of the first parameter onto the aftertreatment system memory. The aftertreatment system controller may utilize the second measurement of the first parameter to determine efficiency of the aftertreatment system or to determine if the intake chamber dosing module and/or decomposition chamber dosing module need to be adjusted to provide a certain amount of treatment fluid.

In some embodiments, the aftertreatment system control strategy may continue to block 728, with determining, by the aftertreatment system controller, a second measurement of the second parameter of the exhaust gas at engine-out. Specifically, a fourth sensor (e.g., fourth sensor 204, fourth sensor 404, fourth sensor 612, etc.) obtains a fourth signal corresponding to the second parameter of the exhaust gas in the intake chamber and provides the fourth signal to the aftertreatment system controller. The aftertreatment system controller determines a second measurement of the second parameter (e.g. NOₓ, SOₓ, particulate concentration, etc.) at engine-out from the provided fourth signal. In some embodiments, the second measurement of the second parameter corresponds to the SOₓ in the exhaust gas in the intake chamber. The aftertreatment system controller may store the second measurement of the second parameter onto the aftertreatment system memory. In some embodiments the fourth sensor is the same as the third sensor. By this way, the aftertreatment system controller may utilize the second measurement of the second parameter to determine the efficiency of the upstream particulate filter and/or the downstream particulate filter. Additionally, the aftertreatment system controller may utilize the second measurement to adjust the first hydrocarbon dosing module (e.g., first hydrocarbon dosing module 148, first hydrocarbon dosing module 348, first hydrocarbon dosing module 548, etc.).

In some embodiments, the aftertreatment system controller may continue to block 730, with determining, by the aftertreatment system controller, whether the intake chamber dosing module and/or the decomposition dosing module is not operating properly. Specifically, the fifth sensor (fifth sensor 206, fifth sensor 406, fifth sensor 614) obtains a fifth signal corresponding to the first parameter of the exhaust gas downstream of the intake chamber and upstream of the decomposition chamber and provides the fifth signal to the after treatment system controller. In some embodiments, the fifth signal corresponds with a second parameter of the exhaust gas downstream of the intake chamber and upstream of the decomposition chamber. The aftertreatment system determines a third measurement of the first parameter from the provided fifth signal. In some embodiments, the third measurement is of the second parameter.

The aftertreatment system controller then compares the third measurement to a third threshold value of the exhaust gas within introduction conduit base. If the third measurement is greater than the third threshold value, then an operator may be notified that the intake chamber dosing module and/or the decomposition chamber dosing module is not operating at a desired rate.

While the aftertreatment system (e.g., aftertreatment system 100, aftertreatment system 300, aftertreatment system 500, etc.) has been shown and described in the context of use with a diesel internal combustion engine, the aftertreatment system may be used with other internal combustion engines, such as gasoline internal combustion engines, hybrid internal combustion engines, propane internal combustion engines, dual-fuel internal combustion engines, and other similar internal combustion engines.

### III. Configuration of Example Embodiments

While this specification contains many specific implementation details, these should not be construed as limitations on the scope of what may be claimed but rather as descriptions of features specific to particular implementations. Certain features described in this specification in the context of separate implementations can also be implemented in combination in a single implementation. Conversely, various features described in the context of a single implementation can also be implemented in multiple implementations separately or in any suitable subcombination. Moreover, although features may be described as acting in certain combinations and even initially claimed as such, one or more features from a claimed combination can, in some cases, be excised from the combination, and the claimed combination may be directed to a subcombination or variation of a subcombination.

As utilized herein, the terms “substantially,” “generally,” “approximately,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the appended claims.

The term “coupled” and the like, as used herein, mean the joining of two components directly or indirectly to one another. Such joining may be stationary (e.g., permanent) or moveable (e.g., removable or releasable). Such joining may be achieved with the two components or the two components and any additional intermediate components being integrally formed as a single unitary body with one another, with the two components, or with the two components and any additional intermediate components being attached to one another.

It is important to note that the construction and arrangement of the various systems shown in the various example implementations is illustrative only and not restrictive in character. All changes and modifications that come within the spirit and/or scope of the described implementations are desired to be protected. It should be understood that some features may not be necessary, and implementations lacking the various features may be contemplated as within the scope of the disclosure, the scope being defined by the claims that follow. When the language “a portion” is used, the item can include a portion and/or the entire item unless specifically stated to the contrary.

Also, the term “or” is used, in the context of a list of elements, in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

Additionally, the use of ranges of values (e.g., W1 to W2, etc.) herein are inclusive of their maximum values and minimum values (e.g., W1 to W2 includes W1 and includes W2, etc.), unless otherwise indicated. Furthermore, a range of values (e.g., W1 to W2, etc.) does not necessarily require the inclusion of intermediate values within the range of values (e.g., W1 to W2 can include only W1 and W2, etc.), unless otherwise indicated.

What is claimed is:

1. An aftertreatment system comprising:
an upstream particulate filter;
a decomposition chamber positioned downstream of the upstream particulate filter;
a decomposition chamber dosing module coupled to the decomposition chamber and configured to provide downstream treatment fluid into the decomposition chamber;
a first downstream catalyst member positioned downstream of the decomposition chamber, the first downstream catalyst member comprising a first downstream catalyst substrate configured to facilitate treatment of exhaust gas; and
a downstream particulate filter positioned downstream of the first downstream catalyst member.

2. The aftertreatment system of claim 1, wherein the downstream particulate filter comprises:
a filter substrate, and
a catalyst coating applied to at least a portion of the filter substrate.

3. The aftertreatment system of claim 1, further comprising:
a first oxidation catalyst member positioned upstream of the upstream particulate filter, the first oxidation catalyst member comprising a first oxidation catalyst housing and a first oxidation catalyst substrate positioned within the first oxidation catalyst housing, the first oxidation catalyst substrate configured to react with hydrocarbons within exhaust gas.

4. The aftertreatment system of claim 3, further comprising:
a first hydrocarbon decomposition chamber positioned upstream of the first oxidation catalyst member; and
a first hydrocarbon dosing module coupled to the first hydrocarbon decomposition chamber and configured to provide hydrocarbons into the first hydrocarbon decomposition chamber.

5. The aftertreatment system of claim 1, further comprising:
an intake chamber positioned upstream of the upstream particulate filter and configured to receive exhaust gas; and
an intake chamber dosing module coupled to the intake chamber, the intake chamber dosing module configured to provide upstream treatment fluid to exhaust gas within the intake chamber.

6. The aftertreatment system of claim 5, further comprising:
an upstream catalyst member positioned upstream of the decomposition chamber and configured to receive exhaust gas, the upstream catalyst member includes an upstream catalyst substrate that comprises vanadia.

7. The aftertreatment system of claim 1, wherein the first downstream catalyst substrate comprises iron zeolite or copper zeolite.

8. The aftertreatment system of claim 1, further comprising:
a downstream ammonia slip catalyst substrate positioned downstream of the first downstream catalyst member and upstream of the downstream particulate filter, the downstream ammonia slip catalyst substrate configured to facilitate treatment of ammonia in exhaust gas.

9. The aftertreatment system of claim 2, wherein:
the catalyst coating comprises:
an inlet coating applied to at least a portion of a surface of a filter substrate inlet of the filter substrate, and
an outlet coating applied to at least a portion of a surface of a filter substrate outlet of the filter substrate.

10. The aftertreatment system of claim 9, wherein:
the inlet coating comprises an iron zeolite catalyst coating, a copper zeolite catalyst coating, or an ammonia slip catalyst coating; and
the outlet coating comprises an iron zeolite catalyst coating, a copper zeolite catalyst coating, or an ammonia slip catalyst coating.

11. The aftertreatment system of claim 9, wherein:
the inlet coating is an iron zeolite catalyst coating; and
the outlet coating is a copper zeolite catalyst coating.

12. The aftertreatment system of claim 1, further comprising:
a second downstream catalyst member positioned downstream of the downstream particulate filter, the second downstream catalyst member comprising a second downstream catalyst substrate configured to facilitate treatment of exhaust gas.

13. The aftertreatment system of claim 1, further comprising:
an upstream catalyst member positioned upstream of the decomposition chamber and configured to receive exhaust gas, the upstream catalyst member includes an upstream catalyst substrate that comprises vanadia.

14. The aftertreatment system of claim 13, further comprising:
an upstream ammonia slip catalyst substrate positioned upstream of the upstream of the decomposition chamber and downstream of the upstream catalyst member;
a third downstream catalyst member positioned downstream of the downstream particulate filter, the third downstream catalyst member a third downstream catalyst substrate configured to facilitate treatment of exhaust gas; and
a downstream ammonia slip catalyst substrate positioned downstream of the third downstream catalyst member.

15. The aftertreatment system of claim 4, further comprising:
a second hydrocarbon decomposition chamber positioned downstream of the decomposition chamber and upstream of the downstream particulate filter;
a second hydrocarbon dosing module coupled to the second hydrocarbon decomposition chamber and configured to provide hydrocarbons into the second hydrocarbon decomposition chamber; and
a second oxidation catalyst fluidly coupled to the second hydrocarbon decomposition chamber.

16. An aftertreatment system comprising:
an intake chamber;
an intake chamber dosing module coupled to the intake chamber and configured to provide an upstream treatment fluid to exhaust gas within the intake chamber;
a decomposition chamber positioned downstream of the intake chamber;
a decomposition chamber dosing module coupled to the decomposition chamber and configured to provide a downstream treatment fluid to exhaust gas within the decomposition chamber;
a downstream particulate filter positioned downstream of the decomposition chamber dosing module;
a first sensor positioned downstream of the downstream particulate filter and configured to provide a first signal; and
an aftertreatment system controller communicable with the first sensor, the intake chamber dosing module, and the decomposition chamber dosing module, the aftertreatment system controller configured to:
cause the intake chamber dosing module to provide a first amount of the upstream treatment fluid into the intake chamber,
cause the decomposition chamber dosing module to provide a second amount of the downstream treatment fluid into the decomposition chamber,
receive the first signal from the first sensor,
determine a first measurement based on the first signal,
determine that the first measurement is greater than a first threshold,
cause the intake chamber dosing module to provide a third amount of the upstream treatment fluid into the intake chamber after determining that the first measurement is greater than the first threshold, the third amount being greater than the first amount, and
cause the decomposition chamber dosing module to provide a fourth amount of the downstream treatment fluid into the decomposition chamber after determining that the first measurement is greater than the first threshold, the fourth amount being less than the second amount and less than the third amount.

17. The aftertreatment system of claim 16, wherein:
the first sensor is configured to provide a second signal;
the aftertreatment system controller is further configured to:
receive the second signal;
determine a second measurement based on the second signal,
determine that the second measurement is greater than a second threshold, and
transmit an adjustment signal associated with an adjustment of an air fuel ratio so as to facilitate regeneration of the downstream particulate filter, after determining that the second measurement is greater than the second threshold.
